(19) United States
(12) Patent Application Publication
(10) Pub. No.: US 2011/0152878 A1
(43) Pub. Date: Jun. 23, 2011

(54) INTERFACE SYSTEMS FOR AIDING CLINICIANS IN CONTROLLING AND MANIPULATING AT LEAST ONE ENDOSCOPIC SURGICAL INSTRUMENT AND A CABLE CONTROLLED GUIDE TUBE SYSTEM

(75) Inventors: Robert M. Trusty, Cincinnati, OH (US); Omar J. Vakharia, Cincinnati, OH (US); Gregory J. Bakos, Mason, OH (US); Kory A. Gunnerson, Cincinnati, OH (US); Jayson G. Moore, Cincinnati, OH (US)

(73) Assignee: Ethicon Endo-Surgery, Inc., Cincinnati, OH (US)

(21) Appl. No.: 12/640,440

(22) Filed: Dec. 17, 2009

(51) Int. Cl. A61B 19/00

(52) U.S. Cl. 606/130

(57) ABSTRACT

Interface systems for interfacing between at least one endoscopic surgical instrument and a cable-controlled guide tube system. Various embodiments include at least one surgical tool docking assembly that is supportable relative to the cable-controlled guide tube system. The surgical tool docking assembly may comprise a cable drive assembly that is operably couplable to the cable-controlled guide system for applying control motions thereto. The surgical tool docking assembly may further comprise at least one tool docking station that is configured to support an endoscopic surgical instrument therein for selective pivotal travel about a first axis and a second axis. The tool docking stations cooperate with corresponding drive shafts for imparting rotary drive motions to the cable drive assembly. Various docking arrangements are disclosed for coupling the cable drive assembly to the cable-controlled guide tube assembly.

### BACKGROUND

The embodiments relate, in general, to endoscopes and medical procedures and, more particularly, to devices for facilitating the insertion and manipulation of endoscopic guide tube assemblies and other surgical instruments within a body cavity to accomplish various surgical and therapeutic procedures.

Minimally invasive procedures are desirable because such procedures can reduce pain and provide relatively quick recovery times as compared with conventional open medical procedures. Many minimally invasive procedures are performed through one or more ports through the abdominal wall, commonly known as trocars. A laparascope that may or may not include a camera may be used through one of these ports for visualization of the anatomy and surgical instruments may be used simultaneously through other ports. Such devices and procedures permit a physician to position, manipulate, and view anatomy, surgical instruments and accessories inside the patient through a small access opening in the patient's body.

Still less invasive procedures include those that are performed through insertion of an endoscope through a natural body orifice to a treatment region. Examples of this approach include, but are not limited to, cystoscopy, hysteroscopy, esophagogastroduodenoscopy, and colonoscopy. Many of these procedures employ the use of a flexible endoscope and flexible or steerable guide tube assemblies during the procedure. Flexible endoscopes often have a flexible, steerable articulating section near the distal end that can be controlled by the user utilizing controls at the proximal end. Treatment or diagnosis may be completed intralumenally, such as polypectomy or gastroscopy. Alternatively, treatment or diagnosis of extra-luminal anatomy in the abdominal cavity may be completed translumenally, for example, through a gastrotomy, colonotomy or vaginotomy. Minimally invasive therapeutic procedures to treat or diagnose diseased tissue by introducing medical instruments translumenally to a tissue treatment region through a natural opening of the patient are known as Natural Orifice Translumenal Endoscopic Surgery (NOTES™).

Regardless of the type of surgery involved and the method in which the endoscope is inserted into the body, the clinicians and surgical specialists performing such procedures have generally developed skill sets and approaches that rely on anatomical alignment for both visualization and tissue manipulation purposes. Over the years, a variety of different endoscope arrangements, as well as various types of steerable sheaths, guide tubes and overtubes for accommodating endoscopes have been developed. For example, various endoscopic guide systems and endoscopes are disclosed in U.S. patent application Ser. No. 12/468,462, entitled “Manipulatable Guide System and Methods For Natural Orifice Translumenal Endoscopic Surgery”, filed May 19, 2009, the disclosure of which is herein incorporated by reference in its entirety. Some of the guide system embodiments disclosed therein include extended articulatable working channels as well as a liftable camera device. Such configurations afford the clinician with the ability to advantageously manipulate and position the working channels while providing the flexibility to position the camera to provide a “bird's eye”, “stadium”, or laparoscopic view of the theater.

While these and other overtube systems and endoscopic surgical devices represent great advancements in the field of Natural Orifice Translumenal Endoscopic Surgery, various surgical procedures require the simultaneous use and manipulation of several of such devices. For example, typical NOTES procedures being done today employ a standard gastroscope through an overtube to gain access and conduct the surgical procedure through the working channels in the gastroscope. The clinician commonly uses one hand to manage the overtube and the second hand to rotate and/or articulate the gastroscope. Other operations might require the use of three or more surgical instruments, making their coordination and precise manipulation challenging. Similarly some overtube arrangements that can articulate in four directions require the clinician to use both hands to operate.

Consequently a need exists for a device that can facilitate the coordinated operation and support of a plurality of endoscopic surgical devices.

The foregoing discussion is intended only to illustrate some of the shortcomings present in the field at the time, and should not be taken as a disavowal of claim scope.

### SUMMARY

In connection with various general forms of the invention there is provided an interface system for aiding clinicians in controlling and manipulating at least one endoscopic surgical instrument and a cable-controlled guide tube system. In connection with various embodiments of the present invention, the interface system may comprise a surgical tool docking assembly that is supportable relative to the cable-controlled guide tube system. The surgical tool docking assembly may comprise a cable drive assembly that is operably couplable to the cable-controlled guide system for applying control motions thereto and a first tool docking station that is configured to support one of the at least one endoscopic surgical instruments for selective pivotal travel about a first axis upon application of a first motion thereto and about a second axis upon application of a second motion thereto. The first tool docking station may be operably coupled to at least one first drive shaft for imparting a corresponding rotary drive motion to the cable drive assembly.

In another general embodiment, there is provided an interface system for aiding clinicians in controlling and manipulating at least one endoscopic surgical instrument and a cable-controlled guide tube system. Various embodiments of the interface system may comprise a central bar that has a first end portion and a second end portion that is spaced from the first end portion. A first tool docking station may be movably coupled to the first end portion of the central bar for selective pivotal travel relative to the central bar about a first axis upon application of a first pivotal motion thereto and a second axis upon application of a second pivotal motion thereto. The first tool docking station may be configured to operably support one of the at least one endoscopic surgical instruments therein. The interface system my further comprise a first friction brake assembly that interacts with the first tool docking station for retaining the first tool docking station in a desired position about the first axis upon discontinuing application of the first pivotal motion to said first tool docking station. A second friction brake assembly may interact with the first tool docking station for retaining the first tool docking station in a desired position about the second axis upon discontinuing application of the second pivotal motion to the first tool docking station. A first cable attachment assembly may be configured to couple a first cable from the cable-controlled guide tube system to the first tool docking station. A second cable attachment assembly may be configured to couple a second cable from the cable-controlled guide tube system to the first tool docking station. A second tool docking station may be movably coupled to the second end portion of the central bar for selective pivotal travel relative to the central bar about a third axis upon application of a third pivot motion thereto and about a fourth axis upon application of a fourth pivotal motion thereto. The second tool docking station may be configured to operably support another one of the at least one endoscopic surgical instruments therein. A third friction brake assembly may interact with the second tool docking station for retaining the second tool docking station in a desired position about the third axis upon discontinuing application of the third pivotal motion to the second tool docking station. A fourth friction brake assembly may interact with the second tool docking station for retaining the second tool docking station in a desired position about the fourth axis upon discontinuing application of the fourth pivotal motion to the second tool docking station. A third cable attachment assembly may be configured to couple a third cable from the cable-controlled guide tube system to the second tool docking station. A fourth cable attachment assembly configured to couple a fourth cable from the cable-controlled guide tube system to the second tool docking station.

In connection with another general embodiment, there is provided a cable docking station for interfacing between a cable drive system and a cable-controlled guide tube system. On connection with various embodiments, the cable docking station may comprise a support member that is configured to dockingly interface with a portion of the cable-controlled guide tube system. A proximal cable coupler may be attached to a distal end of a first cable that extends from the cable drive system. The proximal cable coupler may be operably supported within the support member such that when the support member is docked with the portion of the cable-controlled guide tube system, the proximal cable coupler drivingly engages a corresponding distal cable coupler that is attached to a corresponding first distal cable segment in the cable-controlled guide tube system.

In connection with still another general embodiment of the present invention there is provided an interface system for aiding clinicians in controlling and manipulating at least one endoscopic surgical instrument and a cable-controlled guide tube system. In various embodiments, the interface system comprises a base and a second base that may be rotatably attached to the base for selective rotation relative thereto about a first axis. A first rotator may be rotatably supported on the second base for selective rotation relative thereto about a second axis. The first rotator may be configured to releasably support the endoscopic surgical instrument therein. At least one first steering cable may be attached to the first rotator and coupled to a portion of the cable-controlled guide tube system such that rotation of the first rotator causes said at least one first steering cable to provide at least one actuation motion to the cable-controlled guide tube system.

### BRIEF DESCRIPTION OF THE FIGURES

The novel features of the embodiments described herein are set forth with particularity in the appended claims. The embodiments, however, both as to organization and methods of operation may be better understood by reference to the following description, taken in conjunction with the accompanying drawings as follows.

FIG. 1 is a perspective view of a flexible user interface support assembly embodiment of the present invention supporting two surgical instruments relative to a cable-controlled, steerable guide tube assembly;

FIG. 1A is an enlarged perspective view of a portion of the flexible user interface embodiment of FIG. 1 with one of the surgical instruments removed for clarity;

FIG. 2 is a plan view of the flexible user interface and cable-controlled steerable guide tube assembly of FIG. 1;

FIG. 3 is a front perspective view of a surgical tool docking assembly embodiment of the present invention;

FIG. 4 is a top view of the a surgical tool docking assembly of FIG. 3;

FIG. 5 is a top view of a first tool docking station portion of the surgical tool docking assembly of FIGS. 3 and 4;

FIG. 5A is a cross-sectional view of a portion of a first tool docking station illustrating a first friction brake assembly embodiment of the present invention;

FIG. 6 is a top view of a second tool docking portion of the surgical tool docking assembly of FIGS. 3 and 4;

FIG. 6A is a cross-sectional view of a portion of a second tool docking station illustrating a second friction brake assembly embodiment of the present invention;

FIG. 7 is a partial perspective view of an embodiment of a cable-controlled steerable guide tube assembly;

FIG. 8 is an end view of a portion of the cable-controlled steerable guide tube assembly depicted in FIG. 7;

FIG. 9 is a perspective view of another flexible user interface support assembly embodiment of the present invention with the stand portion omitted for clarity;

FIG. 9A is a cross-sectional view of a portion of a second cable mounting bracket illustrating a friction brake assembly embodiment of the present invention;

FIG. 10 is a top perspective view of the flexible user interface support assembly of FIG. 9, showing the stand portion;

FIG. 11 is a side elevational view of the flexible user interface support assembly of FIG. 9;

FIG. 12 is a partial perspective view of a mounting clamp embodiment of the present invention along with a portion of a surgical instrument;

FIG. 13 is a perspective view of another flexible user interface support assembly of the present invention supporting two endoscopic tools in relation to a steerable guide tube assembly;

FIG. 14 is a rear elevational view of the flexible user interface support assembly depicted in FIG. 13;

FIG. 15 is a partial cross-sectional perspective view of the flexible user interface support assembly depicted in FIGS. 13 and 14;

FIG. 16 is a partial perspective view of a left tool docking station embodiment of the present invention, with a portion of the sphere assembly thereof removed for clarity;

FIG. 17 is a perspective view of a sphere assembly of a left tool docking station embodiment of the present invention;

FIG. 18 is a cross-sectional view of a portion of a left tool docking station embodiment of the present invention:

FIG. 19 is another cross-sectional view of a left tool docking station embodiment wherein the input shaft is in a neutral position;

FIG. 20 is a front elevational view of the left tool docking station embodiment of FIG. 19;

FIG. 21 is a top view of the left tool docking station embodiment of FIGS. 19 and 20;

FIG. 22 is another cross-sectional view of a left tool docking station embodiment wherein the input shaft is in a non-neutral position;

FIG. 23 is a front elevational view of the left tool docking station of FIG. 22;

FIG. 24 is a top view of the left tool docking station of FIGS. 22 and 23;

FIG. 25 is another perspective view of a left tool docking station wherein the input shaft is in a non-neutral position and some components are shown in cross-section for clarity;

FIG. 26 is a partial front elevational view of a left tool docking station embodiment of the present invention;

FIG. 27 is another partial cross-sectional view of a left tool docking station portion of an embodiment of the present invention;

FIG. 28 is another partial cross-sectional view of a left tool docking station embodiment of the present invention;

FIG. 29 is a bottom perspective view of a portion of a right tool docking station embodiment of the present invention;

FIG. 30 is a partial side view of a portion of a right side docking station embodiment of the present invention and a cable mounting assembly embodiment of the present invention;

FIG. 31 is a partial exploded assembly view of a cable mounting assembly embodiment of the present invention;

FIG. 32 is a bottom perspective view of a portion of a cable drive assembly embodiment of the present invention;

FIG. 33 is a bottom perspective view of a cable docking station embodiment of the present invention and a steerable guide tube assembly of the present invention;

FIG. 34 is a perspective assembly view of portions of the cable docking station and the steerable guide tube assembly depicted in FIG. 33;

FIG. 35 is a partial perspective view of the distal end of the flexible sheath portion of the steerable guide tube assembly embodiment of the present invention;

FIG. 36 is a portion of an alternative cable coupling arrangement of the present invention;

FIG. 37 is a side elevation of one cable coupling arrangement of FIG. 36;

FIG. 38 is a partial exploded perspective view of another cable coupling arrangement of the present invention;

FIG. 39 is another partial perspective view of the cable coupling arrangement of FIG. 38 with the housing portions thereof shown in transparent form to illustrate the positions of the internal components;

FIG. 40 is a perspective view of some of the upper and lower rack arrangements of the cable coupling arrangement of FIGS. 38 and 39;

FIG. 41 is a front perspective view of another flexible user interface assembly embodiment of the present invention supporting a portion of an endoscopic surgical instrument;

FIG. 42 is a rear perspective view of the flexible user interface assembly embodiment of FIG. 41;

FIG. 43 is an exploded assembly view of the flexible user interface assembly embodiment of FIGS. 41 and 42;

FIG. 44 is a perspective view of a second base embodiment of the flexible user interface assembly of FIGS. 41-43;

FIG. 45 is a perspective view of a portion of another flexible user interface assembly embodiment of the present invention supporting an endoscopic surgical instrument thereon;

FIG. 46 is a partial exploded assembly view of the flexible user interface assembly of FIG. 45;

FIG. 47 is another partial exploded assembly view of a portion of the flexible user interface assembly of FIGS. 45 and 46; and

FIG. 48 is another partial exploded assembly view of a portion of the flexible user interface assembly of FIGS. 45-47.

### DETAILED DESCRIPTION

U.S. patent application Ser. No. ______, entitled “USER INTERFACE SUPPORT DEVICES FOR ENDOSCOPIC SURGICAL INSTRUMENTS”, Attorney Docket No. END6588USNP/090160 was filed on even date herewith and is owned by the assignee of the present application is herein incorporated by reference in its entirety.

Certain embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting embodiments and that the scope of these embodiments is defined solely by the claims. The features illustrated or described in connection with one embodiment may be combined with the features of other embodiments. Such modifications and variations are intended to be included within the scope of the appended claims.

The various embodiments generally relate to guide systems and steerable sheath arrangements for use in connection with endoscopes for selectively positioning and manipulating endoscopic tools in a desired orientation within the body cavity. The terms “endoscopic tools” and “endoscopic surgical instruments” as used herein may comprise, for example, endoscopes, lights, insufflation devices, cleaning devices, suction devices, hole-forming devices, imaging devices, cameras, graspers, clip appliers, loops, Radio Frequency (RF) ablation devices, harmonic ablation devices, scissors, knives, suturing devices, etc. However, such term is not limited to those specific devices. As the present Description proceeds, those of ordinary skill in the art will appreciate that the unique and novel features of the various instruments and methods for use thereof may be effectively employed to perform surgical procedures by inserting such endoscopic tools through a natural body lumen (mouth, anus, vagina) or through a transcutaneous port (abdominal trocar, cardiothoracic port) to perform surgical procedures within a body cavity.

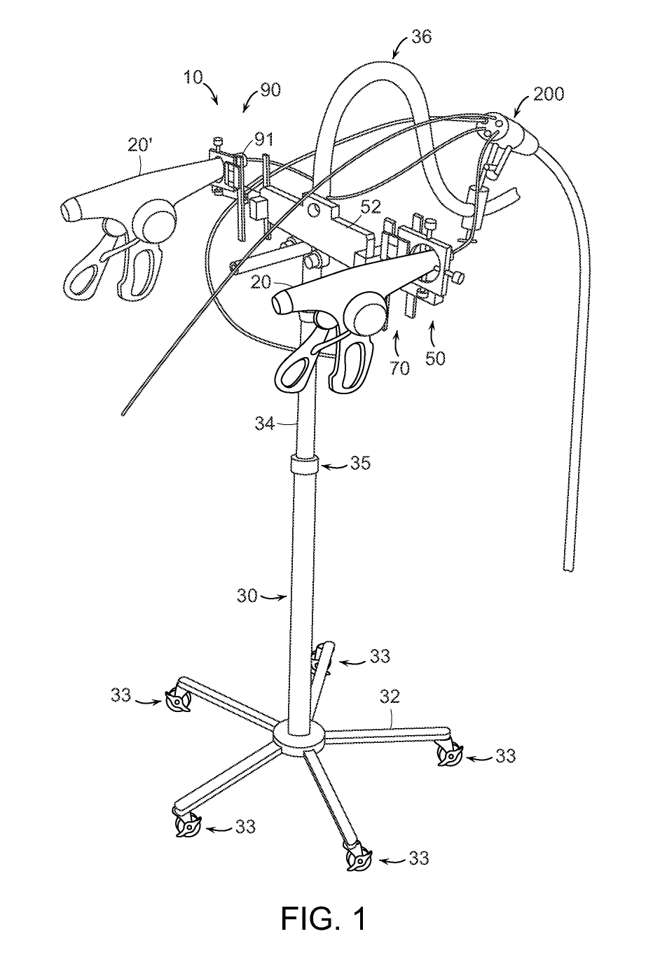

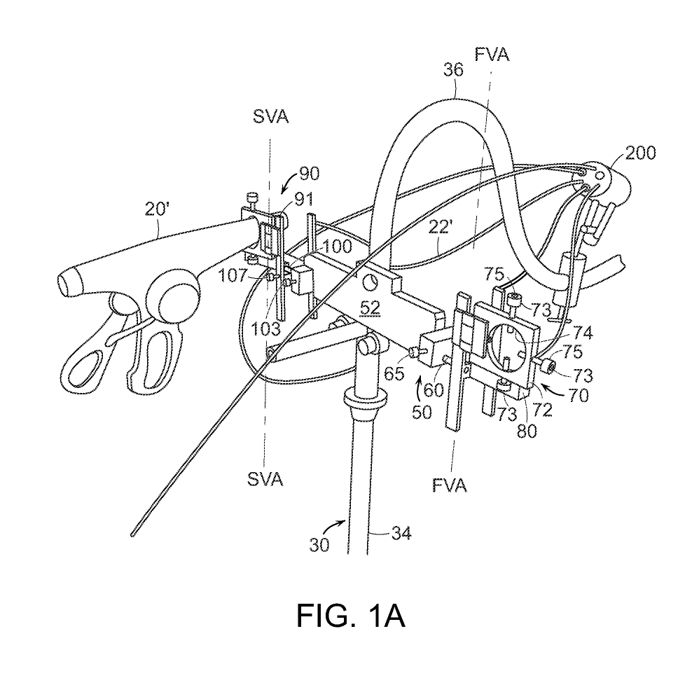

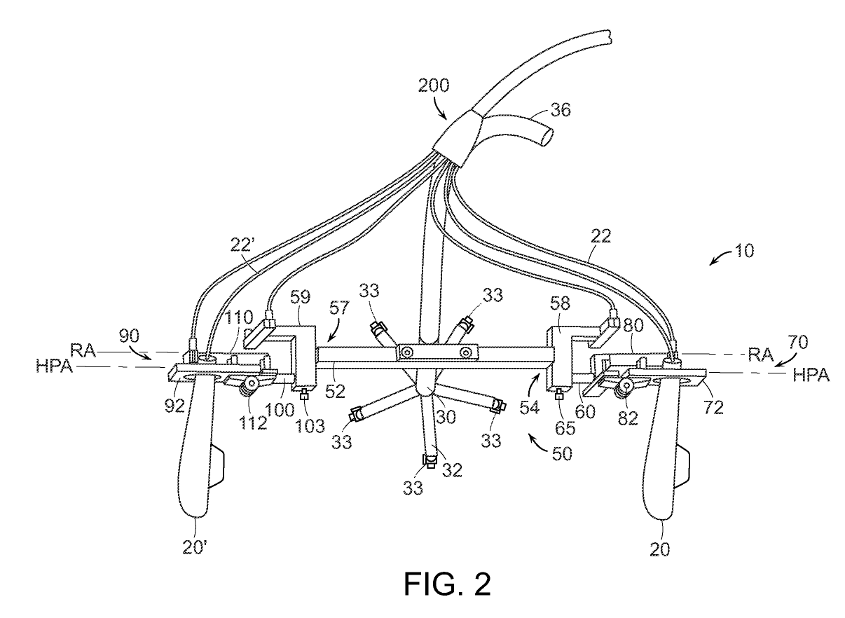

FIGS. 1 and 2 illustrate an embodiment of a flexible user interface support assembly, generally represented as 10, that may operably support two conventional endoscopic surgical instruments 20, 20′. FIG. 1A illustrates the flexible user interface support assembly 10 with only one surgical instrument docked thereto. The surgical instruments 20, 20′ may comprise conventional grasper devices of the type disclosed in U.S. patent application Ser. No. 12/203,330, entitled SURGICAL GRASPING DEVICE, filed Sep. 3, 2008, the disclosure of which is herein incorporated by reference in its entirety. However, the various embodiments of the present invention may be employed with a variety of other types of endoscopic surgical instruments such as, but not limited to, those surgical instruments described above. Accordingly, the scope of protection afforded to the various embodiments disclosed herein should not be limited to their use with a specific type of surgical instrument.

It will be appreciated that the terms “proximal” and “distal” are used herein with reference to a clinician manipulating the surgical instruments 20, 20′. The term “proximal” referring to the portion closest to the clinician and the term “distal” referring to the portion located away from the clinician. It will be further appreciated that for convenience and clarity, spatial terms such as “vertical”, “horizontal”, “up” and “down”, “left” and “right” may be used herein with respect to the drawings. However, surgical instruments are used in many orientations and positions, and these terms are not intended to be limiting and absolute.

As can be further seen in FIGS. 1 and 2, an embodiment of the flexible interface support assembly 10 may include a surgical tool docking assembly 50 that may be operably attached to a stand 30. The stand 30 may comprise a conventional stand that includes a base 32 that has a vertical support bar 34 protruding therefrom. The base 32 may include lockable wheels or casters 33 to facilitate movement of the stand 30. In other embodiments, the stand may comprise an immovable fixture. The vertical support bar 34 of the stand 30 may include a telescopic locknut arrangement 35 to enable the clinician to adjust the vertical height of the mounting assembly 50 to a convenient working height. A flexible “gooseneck” mounting tube 36 may be attached to the top of the vertical support bar 34 and may be selectively positionable in a variety of convenient orientations. Those of ordinary skill in the art will appreciate that the gooseneck mounting tube 36 may be selectively oriented in a variety of different positions/configurations to enable a surgical instrument to be advantageously clamped or otherwise attached thereto as will be explained in further detail below. Such stands are known in the art and, as such, details concerning the specific construction of stand 30 will not be provided herein. For example, those stands manufactured by Anthro of 10450 SW Manhasset Dr., Tualatin, Oreg. under Model No. POC-Cart may be successfully employed. However, those of ordinary skill in the art will understand that the various mounting assembly embodiments of the present invention may be effectively employed with other types of conventional stands without departing from the spirit and scope of the present invention.

Various embodiments of the mounting assembly 50 may include a central cross bar 52 that may be clamped onto or otherwise fastened to the vertical support bar 34 as shown in FIGS. 1, 1A, and 2. It will be further understood, however, that the central cross bar 52 could be attached to a host of other structures in the surgical suite such as the gooseneck mounting tube 36, a table, a bed, etc., without departing from the spirit and scope of the present invention. In various embodiments, the mounting assembly 50 may include a first tool docking station generally depicted as 70 and a second tool docking station, generally depicted as 90. The first tool docking station 70 may be operably attached to a right end 54 of the central cross bar 52 and the second tool docking station may be mounted to the left end 56 of the central cross bar 52. As will be discussed in further detail below, the second tool docking station 90 may be a substantially identical “mirror image” of the first tool docking station 70.

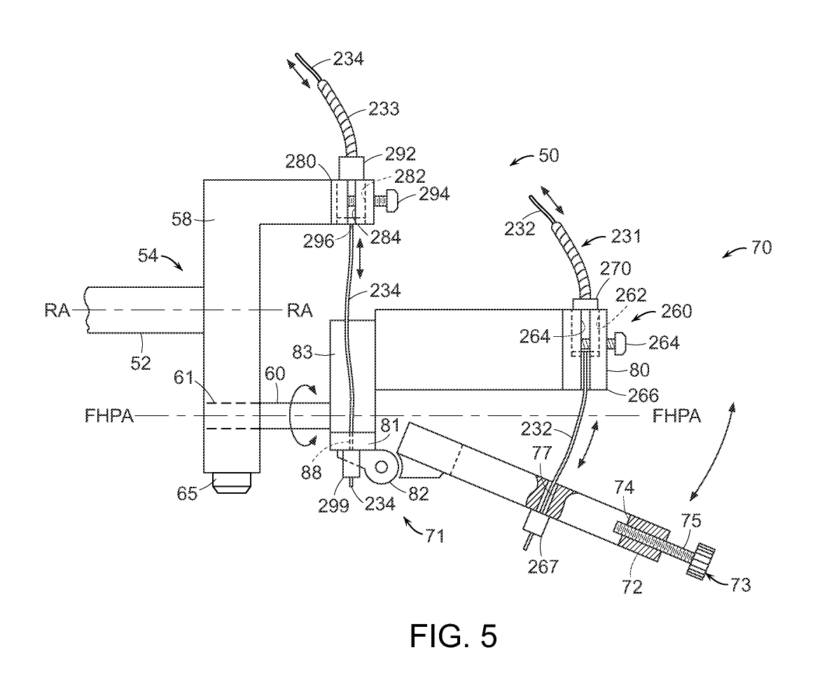

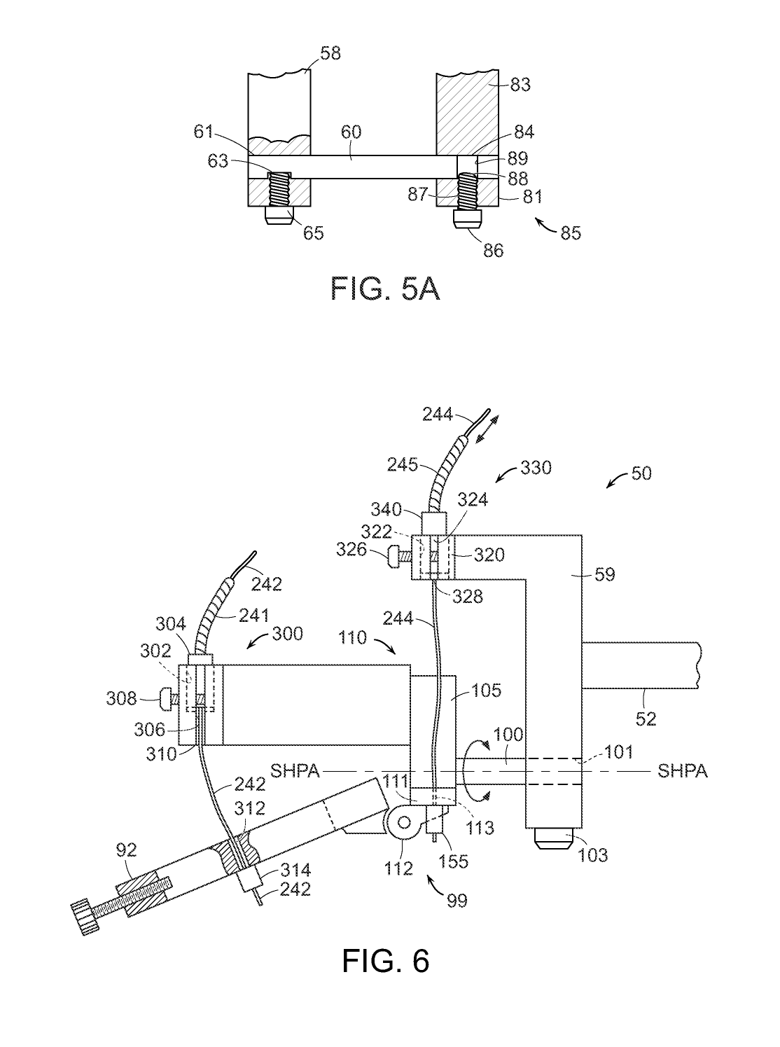

The mounting assembly 50 may comprise a first tool docking station 70 that is mounted for selective movement relative to the central crossbar 52. In various embodiments, a first L-shaped bracket 58 may be attached to the right end 54 of the central crossbar 52. See FIGS. 2 and 5. A first tool mounting bracket 80 may be attached to the first L-shaped bracket 58 by a first pivot bar 60. First pivot bar 60 facilitates selective pivotal travel of the first tool mounting bracket 80 and ultimately the first tool docking station 70 relative to the central crossbar 52 about a first horizontal pivot axis FHPA-FHPA. As can be seen in FIGS. 5 and 5A, the first L-shaped bracket 58 has a hole 61 therethrough for receiving a portion of the first pivot bar 60 therein. The first pivot bar 60 may have a flat surface 63 thereon for engagement with a setscrew 65 as shown in FIG. 5A. The setscrew 65 serves to prevent the first pivot bar 60 from rotating relative to the first L-shaped bracket 58.

Various embodiments may further employ a first horizontal friction brake assembly, generally designated as 85, for controlling the selective pivotal travel of the first tool docking station 70 about the first horizontal pivot axis FHPA-FHPA defined by the first pivot bar 60. As can be seen in FIGS. 5 and 5A, the first tool mounting bracket 80 has a body portion 83 that has a hole 84 for rotatably receiving another end portion of the first pivot bar 60 therein. Thus, the hole 84 is sized relative to the first pivot bar 60 to enable the first pivot bar 60 to rotate therein. In some embodiments, the first friction brake assembly 85 comprises a setscrew 86 that is threaded through a tapped hole 87 in a first vertical mounting plate portion 81 of the first tool mounting bracket 80. See FIG. 5A. The setscrew 86 has a ball 88 on its end that is sized to extend into a groove 89 in the first pivot bar 60. Such arrangement prevents the body portion 83 from translating along the length of the first pivot bar 60 while enabling the ball end 88 of the setscrew 86 to establish a desired amount of frictional engagement with the first pivot bar 60 such that the first tool mounting bracket 80 (and ultimately the first tool docking station 70) is able to rotate about the first pivot bar 60 upon the application of a first amount of pivotal motion to the first tool docking station 70, yet be retained in a desired position after the clinician discontinues the application of the first amount of pivotal motion. Other methods and arrangements for establishing an amount of frictional or braking force between the first tool mounting bracket 80 and the first pivot bar 60 may also be employed. For example, the first friction brake assembly may employ springs, detent arrangements, etc., without departing from the spirit and scope of the present invention.

The first tool docking station 70 may further include a first vertical friction brake assembly, generally designated as 71 for controlling pivotal travel of a first tool docking plate 72 of the first tool docking station 70 about a first vertical axis FVA-FVA. In some embodiments, for example, the first vertical friction brake assembly 71 may comprise a conventional first friction hinge 82 that couples the first tool docking plate 72 to the first tool mounting bracket 80. In particular, the first friction hinge 82 is attached to the first vertical mounting plate portion 81. First friction hinge 82 facilitates selective pivotal travel of the first tool docking plate 72 about the first vertical axis FVA-FVA relative to the first tool mounting bracket 80. For example, those friction hinges manufactured by Reell of 1259 Willow Lake Boulevard, St. Paul Minn. 55110-5103 under Model No. PHC Hinge may be successfully employed. Thus, such arrangement enables the first tool docking station 70 to be selectively pivoted about the first vertical pivot axis FVA-FVA that extends substantially transverse to the first horizontal pivot axis FHPA-FHPA upon application of a second amount of pivotal motion to the first tool docking station and retain the first tool docking station 70 in a desired position about the first vertical pivot axis FVA-FVA when the application of the second amount of pivotal motion to the first tool docking station 70 has been discontinued.

The first tool docking plate 72 is preferably configured to be removably affixed to a first surgical instrument 20. In various embodiments for example, a docking hole 74 may be provided through the first tool docking plate 72 for receiving a portion of the first surgical instrument 20 therethrough. In the embodiment depicted in FIG. 1A, four first docking screws 73 are provided through the first tool docking plate 72 such that the screws 73 are oriented 90 degrees from each other to engage and capture a portion of the first surgical instrument 20 therebetween to removably mount the first surgical instrument 20 to the first tool docking plate 72. At least one, and preferably two, first docking screws 73 may comprise first set screws 75 to enable the clinician to rotate them without the use of tools. See FIG. 1A. Thus, to couple the first surgical instrument 20 to the first docking plate 72, the clinician simply inserts a portion of the first surgical instrument 20 through the hole 74 in the docking plate 72 and then tightens the first set screws 75 in position. Those of ordinary skill in the art will understand, however, that the first tool docking plate 72 may be advantageously configured to retainingly engage a portion of the first surgical instrument 20 so that the first surgical instrument 20 is removably affixed to the first tool docking station 70. For example, the first surgical instrument 20 may be permanently affixed to the first tool docking station 70 by other forms of latches, clamps, etc.

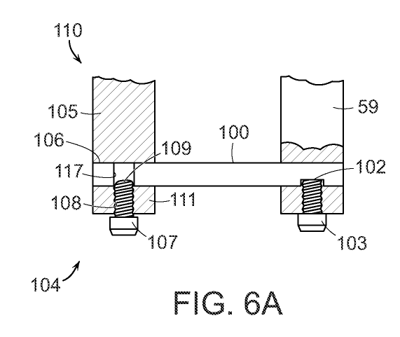

As indicated above and depicted in FIG. 1, the flexible user interface support assembly 10 may be advantageously employed with a second surgical instrument 20′ that may be identical in construction to the first surgical instrument 20 or the second surgical instrument 20′ may comprise an entirely different surgical instrument used to perform entirely different surgical procedures. In this embodiment, the mounting assembly 50 also includes a second tool docking station generally depicted as 90 that may be substantially identical to the first tool docking station 70 and be configured to operably support a second surgical instrument 20′. In various embodiments, a second L-shaped bracket 59 may be attached to the left end 57 of the central crossbar 52. See FIGS. 2 and 6. A second tool mounting bracket 110 may be attached to the second L-shaped bracket 59 by a second pivot bar 100. Second pivot bar 100 facilitates selective pivotal travel of the second tool mounting bracket 110 and ultimately the second tool docking station 90 relative to the central crossbar 52 about a second horizontal pivot axis SHPA-SHPA. In various embodiments, the first horizontal pivot axis FHPA-FHPA may be substantially coaxial with the second horizontal pivot axis SHPA-SHPA and essentially comprise one horizontal pivot axis. As can be seen in FIGS. 6 and 6A, the second L-shaped bracket 59 has a hole 101 therethrough for receiving a portion of the second pivot bar 100 therein. The second pivot bar 100 may have a flat surface 102 thereon for engagement with a setscrew 103 as shown in FIG. 6A. The setscrew 013 serves to prevent the second pivot bar 100 from rotating relative to the second L-shaped bracket 59.

Various embodiments may further employ a second horizontal friction brake assembly, generally designated as 104, for controlling the selective pivotal travel of the second tool docking station 90 about the second horizontal pivot axis SHPA-SHPA defined by the second pivot bar 100. As can be seen in FIGS. 6 and 6A, the second tool mounting bracket 110 has a body portion 105 that has a hole 106 for rotatably receiving another end portion of the second pivot bar 100 therein. Thus, the hole 106 is sized relative to the second pivot bar 100 to enable the second pivot bar 100 to rotate therein. In some embodiments, the second friction brake assembly 104 comprises a setscrew 107 that is threaded through a tapped hole 108 in a second vertical mounting plate portion 111 of the second tool mounting bracket 59. The setscrew 107 has a ball portion 109 that is sized to extend into a groove 117 in the second pivot bar 100. See FIG. 6A. Such arrangement prevents the body portion 105 from translating along the length of the second pivot bar 100 while enabling the ball portion 109 of the setscrew 107 to establish a desired amount of frictional engagement with the second pivot bar 100 such that the second tool mounting bracket 110 (and ultimately the second tool docking station 90) is able to rotate about the second pivot bar 100 upon the application of a third amount of pivotal motion to the second tool docking station 90, yet be retained in a desired position after the clinician discontinues the application of the third amount of pivotal motion. Other methods and arrangements for establishing an amount of frictional or braking force between the second tool mounting bracket 110 and the second pivot bar 100 may also be employed. For example, the second friction brake assembly may employ springs, detent arrangements, etc., without departing from the spirit and scope of the present invention.

The second tool docking station 90 may further include a second vertical friction brake assembly, generally designated as 99 for controlling pivotal travel of a second tool docking plate 92 of the second tool docking station 90 about a second vertical axis SVA-SVA. In some embodiments, for example, the second vertical friction brake assembly 99 may comprise a conventional second friction hinge 112 that couples the second tool docking plate 92 to the second tool mounting bracket 110. In particular, the second friction hinge 112 is attached to a second vertical mounting plate 111 that is attached to the second tool mounting bracket 110. Second friction hinge 112 facilitates selective pivotal travel of the second tool docking plate 92 about a second vertical axis SVA-SVA relative to the second tool mounting bracket 110. Thus, such arrangement enables the second tool docking station 90 to also be selectively pivoted about the second horizontal pivot axis SHPA-SHPA that extends substantially transverse to the second horizontal pivot axis SHPA-SHPA upon application of a fourth amount of pivotal motion to the second tool docking station 90 and retain the second tool docking station 90 in a desired position about the second vertical pivot axis SVA-SVA when the application of the fourth amount of pivotal motion to the second tool docking station 90 has been discontinued.

The second tool docking plate 92 is also preferably configured to be removably affixed to a surgical tool 20′. In various embodiments for example, a docking hole 94 may be provided through the second docking plate 92 for receiving a portion of the surgical instrument 20 therethrough. Four second docking screws 93 are provided through the second tool docking plate 92 that are oriented 90 degrees from each other to engage and capture a portion of the surgical instrument 20′ therebetween to removably mount the surgical instrument 20′ to the second tool docking plate 92. At least one and preferably two first docking screws 93 may comprise second set screws 95 to enable the clinician to rotate them without the need of tools.

To couple the second surgical instrument 20′ to the second tool docking plate 92, the clinician simply inserts a portion of the second surgical instrument 20′ through the hole 94 in the second tool docking plate 92 and then tightens the second set screws 95 in position. Those of ordinary skill in the art will understand, however, that the second tool docking plate 92 may be advantageously configured to retainingly engage a portion of the second surgical instrument 20′ so that the second surgical instrument 20′ is removably affixed to the second tool docking station 90. For example, the second surgical instrument 20′ may be removably affixed to the second tool docking station 90 by other forms of latches, clamps, etc. In various embodiments, the tool docking assembly may be manufactured from steel, aluminum, stainless steel, or plastic and may be of welded construction or the various bracket portions thereof may comprise separate components that are interconnected with suitable fasteners such as screws, bolts etc.

The flexible user interface support assembly 10 may be advantageously employed with a cable-controlled, steerable guide tube assembly 200 which may be supported, for example, by the gooseneck mounting tube 36. Various forms of steerable guide tube assemblies are known. For example, the various embodiments of the present invention may be successfully used in connection with various cable actuated manipulatable guide systems disclosed in U.S. patent application Ser. No. 12/468,462, filed May 19, 2009, entitled “MANIPULATABLE GUIDE SYSTEM AND METHODS FOR NATURAL ORIFICE TRANSLUMENAL ENDOSCOPIC SURGERY”, the disclosure of which is herein incorporated by reference in its entirety.

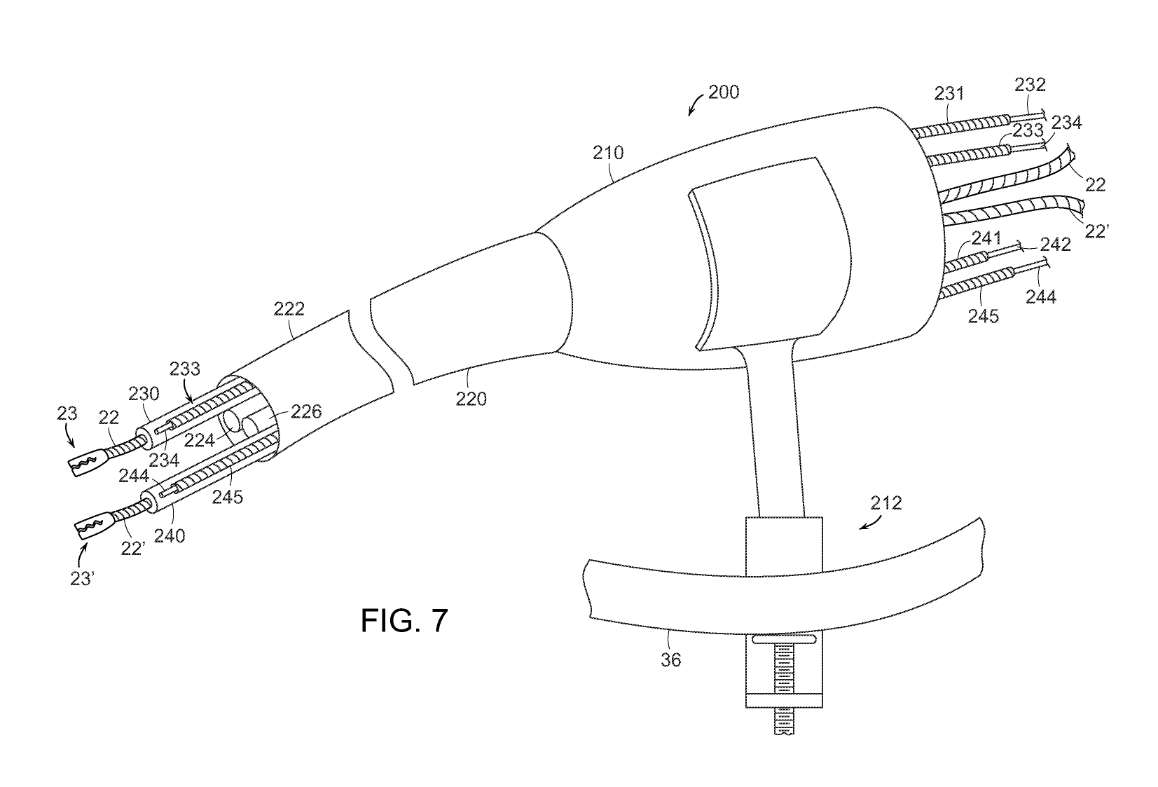

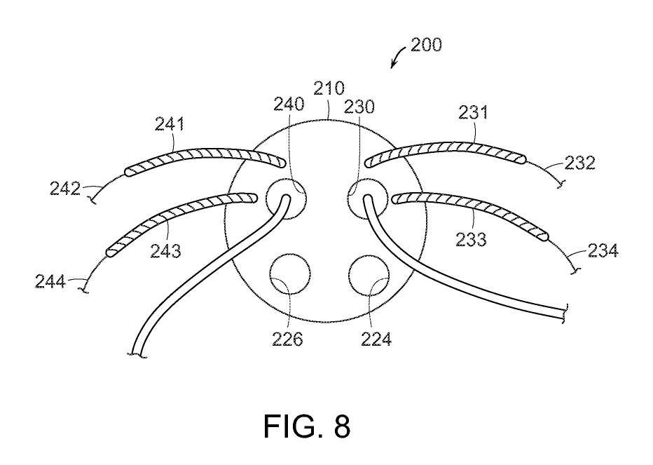

As can be seen in FIGS. 1 and 7, the steerable guide tube assembly 200 may comprise a handle portion 210 that may be clamped or otherwise attached to the gooseneck mounting tube 36 by a clamp 212 that facilitates removal and repositioning of the handle 210 on the gooseneck mounting tube 36. An inner sheath assembly 220 is attached to and protrudes from the handle 210 for insertion into the patient, through, for example, a natural orifice or other access opening made in the patient. As discussed in the aforementioned patent application, the inner sheath assembly 220 may comprise an inner sheath 222 that supports at least one and preferably a plurality of working channels 230, 240 therein. For example, in the embodiment depicted in FIG. 6, the inner sheath 222 supports a selectively positionable right working channel 230 and a selectively positionable left working channel 240 therein. The inner sheath 222 may also support various other working channels 224, 226, etc. therein that may be selectively positionable or may simply comprise flexible lumens supported within the inner sheath assembly 222. In use, for example, a flexible working portion 22 of the surgical instrument 20 may extend through one of the working channels 230, 240 such that the distal tip 23 thereof may be selectively positioned within the patient by steering the working channel through which it extends. See FIG. 7. Similarly, the flexible working portion 22′ of the surgical instrument 20′may extend through one of the working channels 230, 240 such that the distal tip 23′ thereof may be selectively positioned within the patient by steering the working channel through which it extends.

By way of example, however, in various embodiments, the right working channel 230 is controlled by a first “left/right” articulation cable 232 and a first “up/down” articulation cable 234. The first left/right articulation cable 232 may extend through a flexible cable sheath or coil tube 231 that extends through the inner sheath assembly 222 and the first up/down articulation cable 234 may extend through a flexible coil tube or cable sheath 233 that extends within the inner sheath assembly 222. In various embodiments, the first left/right articulation cable 232 is sized relative to the flexible coil tube 231 such that it is freely movable therein. Similarly, the first up/down cable 234 is sized relative to the flexible coil tube 233 such that it is freely movable therein. Also in various embodiments, the left working channel 240 is controlled by a “left/right” articulation cable 242 that is received within a flexible cable sheath or coil tube 241 that extends through the inner sheath assembly 222. The second left/right articulation cable 242 is sized relative to the flexible coil tube 241 such that it is freely movable therein. The left working channel 240 may be further controlled by an “up/down” articulation cable 244 that is received in a flexible cable sheath or coil tube 245 that extends through the inner sheath assembly 222. The second up/down articulation cable 244 is sized relative to the coil tube 245 such that it is freely movable therein. In various embodiments, the articulation cables 232, 234, 242, 244 and their respective coil tubes 232, 233, 241, 243 extend proximally out through the handle portion 210 of the steerable guide tube assembly 200 and are adapted to be coupled to the user interface support assembly 10 to enable the selectively positionable right and left working channels 230 and 240 to be moved automatically in response to the manipulation of the surgical instruments 20, 20′, respectively.

Various embodiments of the present invention may employ quick-connection arrangements for coupling the cables 232, 234, 242, 244 and their respective coil tubes 231, 233, 241, 243 to the mounting assembly 50. Various methods for attaching the first articulation cables 232 and 234 to the mounting assembly 50 are depicted in FIG. 5. As can be seen in that Figure, the first left/right articulation cable 232 and its coil tube 231 may be operably coupled to the mounting assembly 50 by a first cable attachment assembly generally designated as 260. The first cable attachment assembly 260 may comprise a bore 262 that is provided in the first tool mounting bracket 80 and is sized to receive therein a ferrule 270 that is attached to the coil tube 231 of the first left/right articulation cable 232. The first tool mounting bracket 80 may further have a slit 264 that extends into the bore 262 such that when the ferrule 270 is inserted into the bore 262, it can be retained therein by a set screw 264. The first left/right articulation cable 232 passes through a smaller diameter hole 266 in the first tool mounting bracket 80 and is inserted through a hole 77 in the first tool docking plate 72. The end of the first left/right articulation cable 232 is affixed to the first tool docking plate 72 by a tube segment 267 that is crimped onto or otherwise affixed to the end of the cable 232 and which has a diameter that is larger than hole 73 in the tool docking plate 72. Thus, by pivoting the first tool docking plate 72 about the first vertical axis FVA-FVA, the clinician can actuate the first left/right articulation cable 232 to cause the first working channel 230 to articulate in a left or right direction depending upon whether the cable 232 is being pushed through the coil tube 231 or pulled through the coil tube 231. In particular, when the clinician pivots the first surgical tool 20 and the first tool docking plate 72 about the first vertical axis FVA-FVA in a direction towards the first tool docking bracket 80, the first left right articulation cable is pushed through the coil tube 231 and the distal end of the first working channel 230 is articulated to a “first” or left direction. When the clinician moves the first surgical tool 20 and the first tool docking plate 72 away from the first tool docking bracket 80, the first left/right articulation cable 232 is pulled through the coil tube 231 and the distal end of the first working channel is articulated to a “second” or right direction.

Also in various embodiments, the first up/down articulation cable 234 is attached to the first tool mounting bracket 80 and a first cable standoff plate 280 that is attached to the first L-shaped bracket 58 by a second cable attachment assembly generally designated as 290. The second cable attachment assembly 290 may comprise a bore 282 that is provided in the first cable standoff plate 280 and is sized to receive therein a ferrule 292 that is attached to the outer sheath 233 of the first up/down articulation cable 234. The first cable standoff plate 280 may further have a slit 284 that extends into the bore 282 such that when the ferrule 292 is inserted into the bore 282, it can be retained therein by a set screw 294. The cable 234 passes through a smaller diameter hole 296 in the first cable standoff plate 280 and is inserted through a hole 88 in the first vertical mounting plate 81. The end of the cable 234 is affixed to the first vertical mounting plate 81 by a tube segment 299 that is crimped onto or otherwise affixed to the end of the cable 234 and which has a diameter that is larger than hole 88 in the first vertical mounting plate 81. Thus, by pivoting the first mounting bracket 80 and the first vertical mounting plate 81 attached thereto about pivot axis PA-PA, the clinician can actuate the first up/down cable 234 to cause the distal end of the first working channel 230 to articulate up and down depending upon whether the cable 234 is being pushed through the coil tube 233 or pulled through the coil tube 233. For example, when the clinician pivots first surgical tool 20 and the first tool docking plate 72 in a direction towards the steerable guide tube assembly 200 about the horizontal pivot axis HPA-HPA, the first up/down articulation cable 234 is pushed through the coil tube 233 which causes the distal end of the first working channel to pivot downward. Likewise, when the clinician pivots the first surgical tool 20 and the first tool docking plate 72 away from the steerable guide tube assembly 200 about horizontal pivot axis HPA-HPA, the distal end of the first working channel 230 is articulated in an upward direction.

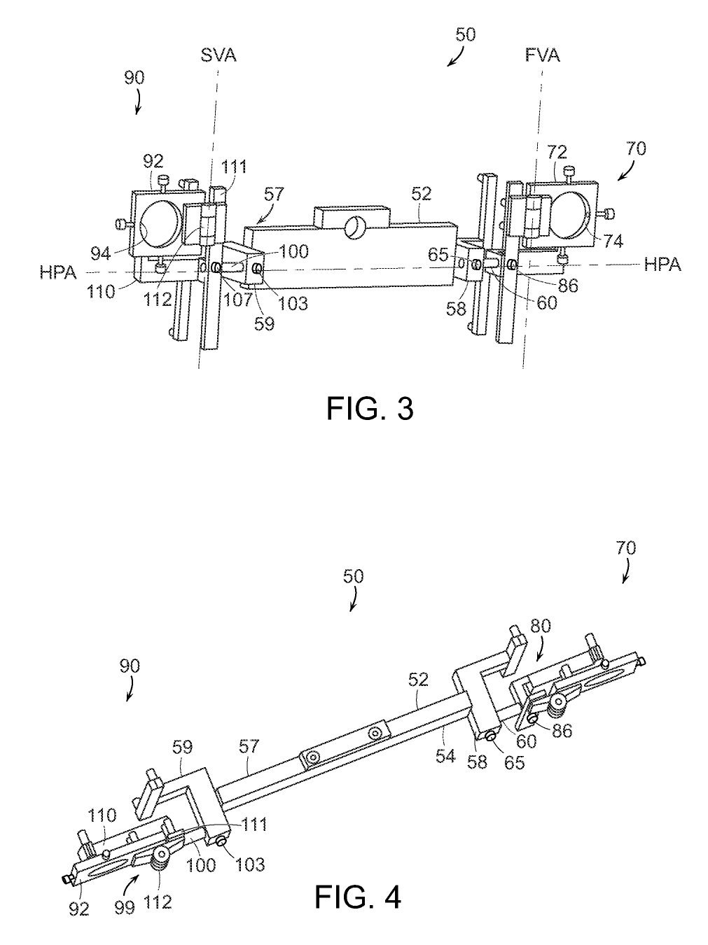

As can be seen in FIG. 6, the second left/right articulation cable 242 and its coil tube 241 may be operably coupled to the mounting assembly 50 by a third cable attachment assembly generally designated as 300. The third cable attachment assembly 300 may comprise a bore 302 that is provided in the second tool mounting bracket 110 and is sized to receive therein a ferrule 304 that is attached to the coil tube 241 of the second left/right articulation cable 242. The second tool mounting bracket 110 may further have a slit 306 that extends into the bore 302 such that when the ferrule 304 is inserted into the bore 302, it can be retained therein by a set screw 308. The second left/right articulation cable 242 passes through a smaller diameter hole 310 in the second tool mounting bracket 110 and is inserted through a hole 312 in the second tool docking plate 92. The end of the second left/right articulation cable 242 is affixed to the second tool docking plate 92 by a tube segment 314 that is crimped onto or otherwise affixed to the end of the cable 242 and which has a diameter that is larger than hole 312 in the second tool docking plate 92. Thus, by pivoting the second tool docking plate 92 about the second vertical axis SVA-SVA (FIG. 3), the clinician can actuate the second left/right articulation cable 242 to cause the second working channel 240 to articulate in a left or right direction depending upon whether the cable 242 is being pushed through the coil tube 241 or pulled through the coil tube 241. In particular, when the clinician pivots the second surgical tool 20′ which, in turn, pivots the second tool docking plate 92 about the second vertical axis SVA-SVA in a direction towards the second tool docking bracket 110, the second left/right articulation cable 242 is pushed through the coil tube 241 and the distal end of the second working channel 240 is articulated to a “third” or left direction. When the clinician moves the second surgical tool 20′ and the second tool docking plate 92 away from the second tool docking bracket 110, the second left/right articulation cable 242 is pulled through the coil tube 241 and the distal end of the second working channel 240 is articulated to a “fourth” or right direction.

Also in various embodiments, the second up/down articulation cable 244 is attached to the second tool mounting bracket 110 and a second cable standoff plate 320 that is attached to the second L-shaped bracket 59 by a fourth cable attachment assembly generally designated as 330. The fourth cable attachment assembly 330 may comprise a bore 322 that is provided in the second cable standoff plate 320 and is sized to receive therein a ferrule 340 that is attached to the coil tube 245 of the second up/down articulation cable 244. The second cable standoff plate 320 may further have a slit 324 that extends into the bore 322 such that when the ferrule 340 is inserted into the bore 322, it can be retained therein by a set screw 326. The cable 244 passes through a smaller diameter hole 328 in the second cable standoff plate 320 and is inserted through a hole 113 in the second vertical mounting plate 111. The end of the cable 244 is affixed to the second vertical mounting plate 111 by a tube segment 115 that is crimped onto or otherwise affixed to the end of the cable 244 and which has a diameter that is larger than hole 113 in the second vertical mounting plate 111. Thus, by pivoting the second mounting bracket 110 and the second vertical mounting plate 111 attached thereto about horizontal pivot axis HPA-HPA, the clinician can actuate the second up/down articulation cable 244 to cause the distal end of the second working channel 240 to articulate up and down depending upon whether the cable 244 is being pushed through the coil tube 245 or pulled through the coil tube 245. For example, when the clinician pivots the second surgical tool 20′ and the second tool docking plate 92 in a direction towards the steerable guide tube assembly 200 about the horizontal pivot axis HPA-HPA, the second up/down articulation cable 244 is pushed through the coil tube 245 which causes the distal end of the second working channel 240 to pivot downward. Likewise, when the clinician pivots the second surgical tool 20′ and the second tool docking plate 92 away from the steerable guide tube assembly 200 about horizontal pivot axis HPA-HPA, the distal end of the second working channel 240 is articulated in an upward direction.

While the above-described embodiments are configured to support two endoscopic surgical instruments, those of ordinary skill in the art will understand that various embodiments of the present invention may be constructed to support a single instrument or more than two instruments. It will be further appreciated that depending upon how the cables are attached to the respective tool docking stations 70, 90, movement of the handle portions of the surgical instruments 20, 20′causes the cable controlled guide tube to impart laparoscopic-like movement of the distal tip of the flexible portion of the surgical instrument. For example, when the handle is lifted up, the cable controlled working channel through which the flexible working portion extends may move the tip portion downward or upward depending upon how the cables are coupled to the tool docking stations. Likewise, when the handle is moved left, the working channel may cause the distal tip to move left or right. It will be further appreciated that the unique and novel features of the various embodiments of the interface system 10 of the present invention enable the control cables for the cable controlled guide tube system to remain in any desired fixed position after the pivotal motions applied to the tool docking stations or the surgical instruments docked therein have been discontinued.

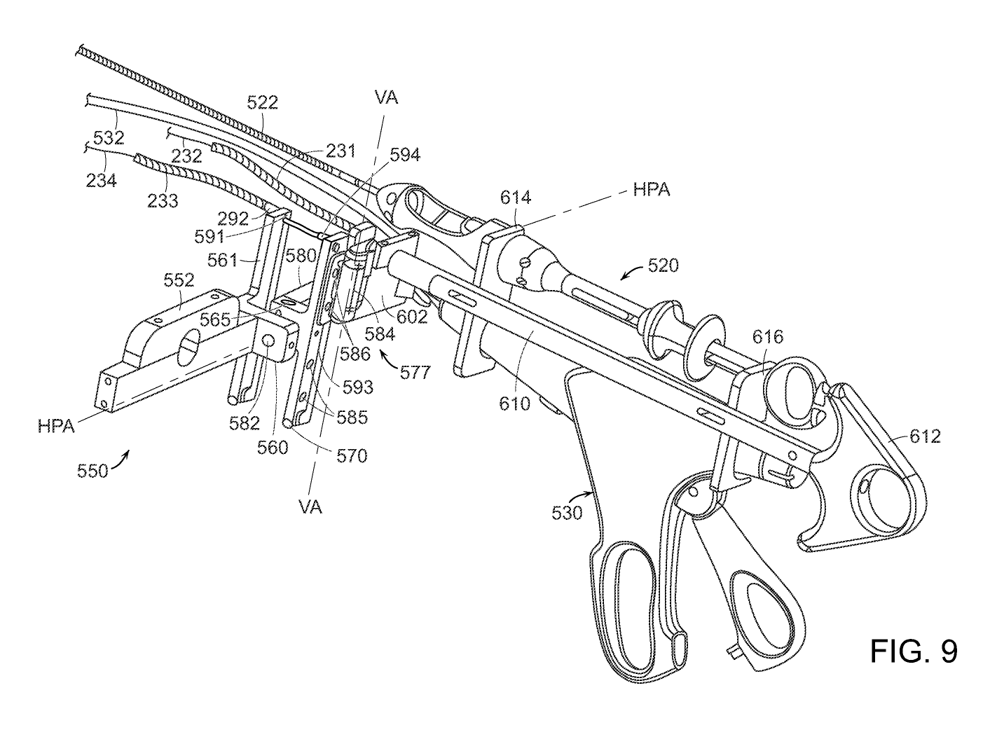

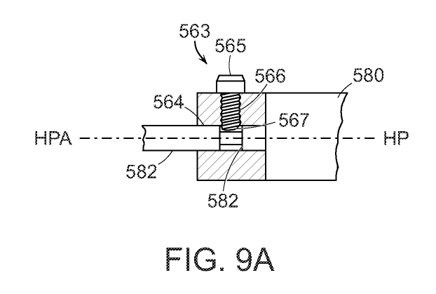

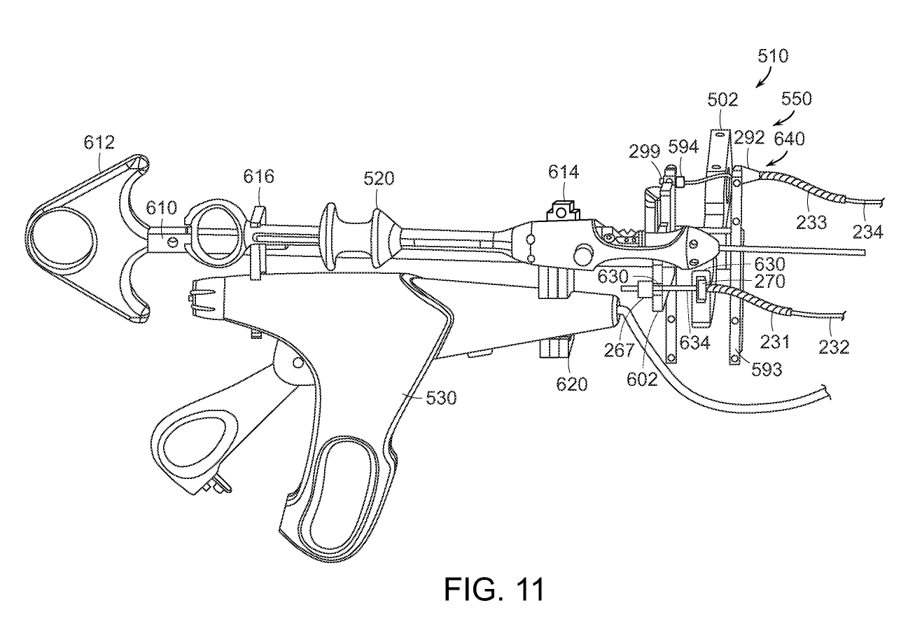

FIGS. 9-11 depict another flexible interface support assembly 510 of the present invention that is adapted for use in connection with two endoscopic surgical instruments 520, 530. In the depicted embodiment, for example, surgical instrument 520 may comprise a conventional clip application device and surgical instrument 530 may comprise a conventional grasping device of the construction described above. One form of clip application device is disclosed in U.S. patent application Ser. No. 12/172,766, filed Jul. 14, 2008, and entitled TISSUE APPOSITION CLIP APPLICATION DEVICES AND METHODS, the disclosure of which is herein incorporated by reference in its entirety. Other forms of surgical instruments may be effectively employed with the various embodiments of the present invention disclosed herein. Other of such instruments are disclosed for example in U.S. patent application Ser. No. 12/133,109, filed Jun. 4, 2008, entitled “ENDOSCOPIC DROP OFF BAG”; U.S. patent application Ser. No. 11/610,803, entitled “MANUALLY ARTICULATING DEVICES”; and U.S. patent application Ser. No. 12/170,126, entitled “DEVICES AND METHODS FOR PLACING OCCLUSION FASTENERS”, the respective disclosures of which are herein incorporated by reference in their entireties.

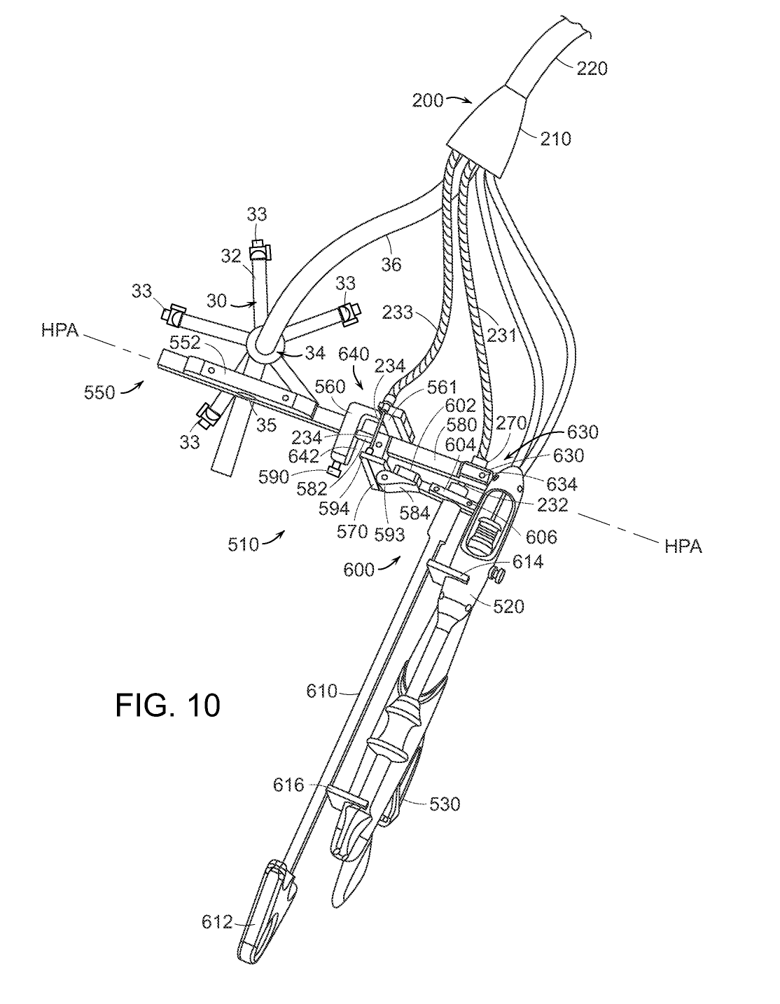

Various embodiments of the flexible user interface support assembly 510 may include a stand mounting bracket 550 that may be attached to a stand 30 of the type and construction described above. The stand mounting bracket 550 may include a clamp portion 552 that can be removably clamped onto a horizontal mounting rod 35 attached to the stand 30. See FIG. 10. However, other clamping and fastener arrangements may be employed to affix the stand mounting bracket 550 to the stand 30 without departing from the spirit and scope of the present invention. As can be seen in FIGS. 9-11, a first, L-shaped cable mounting bracket 560 may be attached to the stand mounting bracket 550. The first cable mounting bracket 560 may include a vertically extending section 561 to enable a first cable outer jacket end ferrule 292 to be mounted thereto.

As can be further seen in FIGS. 9-11, a second substantially “T-shaped” cable mounting bracket 570 may be pivotally attached to a first cable mounting bracket 560 by a pivot rod 582 that facilitates pivotal travel of the second cable mounting bracket 570 (and a tool docking assembly 600 attached thereto) relative to the first cable mounting bracket 560 about a horizontal pivot axis HPA-HPA defined by pivot rod 582. The pivot rod 582 may be non-rotatably attached to the first cable mounting bracket 560 by a set screw 590. Various embodiments may also employ a friction brake assembly, generally designated as 563, for controlling the selective pivotal travel of the second cable mounting bracket 570 and the tool docking assembly 600 attached thereto about the horizontal pivot axis HPA-HPA. As can be seen in FIG. 9A, a hole 564 is provided through the second cable mounting bracket 570 for rotatably receiving another end portion of the pivot rod 582 therein. Thus, the hole 564 is sized relative to the pivot rod 582 to enable the pivot rod 582 to rotate therein. In some embodiments, the friction brake assembly 563 comprises a setscrew 565 that is threaded through a tapped hole 566 in the second cable mounting bracket 570. The setscrew 565 has a ball 567 thereon that is sized to extend into a groove 568 in the pivot rod 582. Such arrangement prevents the second cable mounting bracket 570 from translating along the length of the pivot rod 582 while enabling the ball end 567 of the setscrew 565 to establish a desired amount of frictional engagement with the pivot rod 582 such that the second cable mounting bracket 570 (and ultimately the tool docking assembly 600) is able to rotate about the pivot rod 582 upon the application of a first amount of pivotal motion to the tool docking assembly 600, yet be retained in a desired position after the clinician discontinues the application of the first amount of pivotal motion. Other methods and arrangements for establishing an amount of frictional or braking force between the second cable mounting bracket 570 and the pivot rod 582 may also be employed. For example, the first friction brake assembly may employ springs, detent arrangements, etc., without departing from the spirit and scope of the present invention.

Also in various embodiments, a third cable mounting bracket 591 may be connected to the first cable mounting bracket 560 to releasably trap the cable outer jacket ferrule 292 in a loose, pivotable manner while allowing cable 234 to translate freely therein. A fourth cable mounting bracket 593 may be mounted to the second cable mounting bracket 570 to pivotally lock the ferrule 594 at the end of the cable 234 between the second cable mounting bracket 570 and the fourth cable mounting bracket 593. When configured as described above, a downward pivoting of the tool 530 will cause the second cable mounting bracket 570 and fourth cable mounting bracket 593 to pivot about pin 582 and pull cable 234 within a locked outer jacket 233 to facilitate motion of the cable at the interface between the assembly 10 and cable controlled guide-tube system. Pivotable mounting of the outer cable jacket end ferrule 292 and cable end ferrule 594 allows use of a solid core cable without bending or kinking.

The tool docking assembly 600 may further include a vertical friction brake assembly, generally designated as 577 for controlling pivotal travel of the tool docking assembly 600 about a vertical axis VA-VA. In some embodiments, for example, the vertical friction brake assembly 577 may comprise a conventional friction hinge 584 that couples a tool mounting docking plate 602 to the second cable mounting bracket 570. In particular, the friction hinge 584 is attached to the second cable mounting bracket 570. In various embodiments, the second cable mounting bracket 570 may be provided with a plurality of threaded mounting holes 585 to accommodate fastening of a friction hinge 584 thereto to accommodate different surgical tool arrangements. Such arrangement enables the tool docking assembly 600 to be selectively pivoted about the vertical pivot axis VA-VA that extends substantially transverse to the horizontal pivot axis HPA-HPA upon application of a second amount of pivotal motion to the tool docking assembly 600 and retain the tool docking assembly 600 in a desired position about the vertical pivot axis VA-VA when the application of the second amount of pivotal motion to the tool docking assembly 600 has been discontinued.

Various embodiments of the tool docking assembly 600 may include a input shaft 610 that is attached to the tool docking plate 602 by a clamp feature 604 and set screws 606. Attached to the input shaft 610 is a pair of mounting clamps 614, 616 that are configured to engage and support the surgical instruments 520, 530. An ergonomic handle 612 may be provided on the proximal end of the input shaft 610 to facilitate pivoting of the input shaft 610 and surgical tools 520, 530 mounted thereto about vertical axis VA-VA.

As show in FIG. 10, the flexible interface support assembly 510 may be employed in connection with a cable-controlled, steerable guide tube assembly 200 which may be supported, for example, by the gooseneck mounting tube 36. In this embodiment, the inner sheath assembly 222 that protrudes from handle portion 210 includes at least one steerably working channel 230 of the type described above, the distal end of which may be articulated in the left/right directions and in the up/down directions. In particular, a left/right articulation cable 232 is attached to the distal end of the working channel 230 as was described above and depicted, for example, in FIG. 6. The left/right articulation cable 232 may extend through a flexible cable sheath or coil tube 231 that extends through the inner sheath assembly 222. In various embodiments, the left/right articulation cable 232 is sized relative to the flexible coil tube 231 such that it is freely movable therein.

The left/right articulation cable 232 and its coil tube 231 may be operably coupled to the tool docking assembly 600 by a first quick-connection arrangement generally designated as 630. The quick-connection arrangement 630 may comprise a clamp feature 632 and set screw 634 that is provided in the second cable mounting bracket 580 and is configured to clamp a ferrule 270 that is attached to the coil tube 231 of the left/right articulation cable 232. The left/right articulation cable 232 passes through a smaller diameter hole 634 in the second cable mounting bracket 280 and is inserted through a hole 636 in the tool docking plate 602. The end of the left/right articulation cable 232 is affixed to the tool docking plate 602 by an end ferrule 267 that is crimped onto or otherwise affixed to the end of the cable 232 and which has a diameter that is larger than hole 636 in the tool docking plate 602.

Pivoting the tool docking plate 602 about the pin axis (vertical axis VA-VA) of the friction hinge 584 results in the cable 232 translating within the cable outer jacket 231 to facilitate motion of the cable 232 at the interface between the assembly 10 and the cable-controlled guide tube system. Pivotable mounting of the outer cable jacket end ferrule 270 and the cable end ferrule 267 allows the use of a solid core cable without bending or kinking. Such arrangement enables the clinician to actuate the left/right articulation cable 232 to cause the first working channel 230 to articulate in a left or right direction depending upon whether the cable 232 is being pushed through the coil tube 231 or pulled through the coil tube 231. In particular, when the clinician pivots the tool docking plate 602 about the vertical axis VA-VA in a direction towards the second tool docking bracket 580, the left/right articulation cable 232 is pushed through the coil tube 231 and the distal end of the first working channel 230 is articulated to a “first” or left direction. When the clinician moves the tool docking plate 602 away from the second tool docking bracket 580, the left/right articulation cable 232 is pulled through the coil tube 231 and the distal end of the first working channel 230 is articulated to a “second” or right direction.

Also in various embodiments, the up/down articulation cable 234 is attached to the first vertically extending cable mounting bracket 570 and a second vertically extending cable mounting plate 583 that is attached to the second cable mounting bracket 580 by a second quick-connection arrangement generally designated as 640. The second quick-connection arrangement 640 may comprise a bore that is provided in the first vertically extending cable mounting bracket 570 and is sized to receive therein a ferrule 292 that is attached to the outer sheath 233 of the up/down articulation cable 234. The ferrule 292 may be held in position by a clamping feature or other arrangement. The cable 234 passes through the first vertically extending cable mounting bracket 570 and is inserted through a hole 642 in the second vertically extending cable mounting plate 583. The end of the cable 234 is affixed to the second vertically extending cable mounting plate 583 by a tube segment 299 that is crimped onto or otherwise affixed to the end of the cable 234 and which has a diameter that is larger than hole 642 in the second vertically extending cable mounting plate 583.

By pivoting the second cable mounting bracket 580 and the second vertically extending cable mounting plate 583 attached thereto about pivot axis PA-PA, the clinician can actuate the up/down cable 234 to cause the distal end of the working channel 230 to articulate up and down depending upon whether the cable 234 is being pushed through the coil tube 233 or pulled through the coil tube 233. For example, when the clinician pivots first the second cable mounting bracket 580 and the second vertically extending cable mounting plate 583 in a direction towards the steerable guide tube assembly 200 about the horizontal pivot axis HPA-HPA, the up/down articulation cable 234 is pushed through the coil tube 233 which causes the distal end of the first working channel 230 to pivot downward. Likewise, when the clinician pivots the second cable mounting bracket 580 and the second vertically extending cable mounting plate 583 away from the steerable guide tube assembly 200 about horizontal pivot axis HPA-HPA, the distal end of the first working channel 230 is articulated in an upward direction. Those of ordinary skill in the art will appreciate that either or both of the flexible sheath portions 522, 532 of the surgical instruments, respectively may be inserted through the first working channel 230 or only one of those sheaths 522, 532 may be inserted through the working channel 230 and the other sheath may be inserted through another working channel in the guide tube assembly 200.

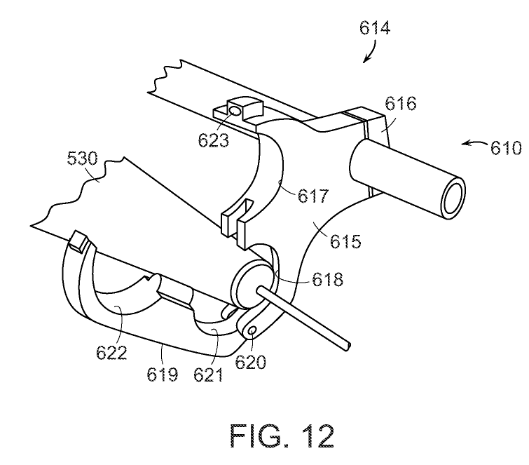

In various embodiments, the endoscopic surgical instruments 520, 530 may be releasably coupled to the input shaft 610 by a clamp 614. As can be seen in FIG. 12, the clamp 614 may comprise a clamp body 615 that has a first clamp arm 616 that may be attached thereto by screws (not shown) to facilitate clamping of the clamp body 615 to the input shaft 610 as shown in FIG. 12. The clamp body 615 may be provided with a plurality of tool docking station recesses 617, 618 that are sized to receive a portion of the endoscopic surgical instruments 520, 530 therein. A second clamp arm 619 may be attached to the clamp body 615 by a hinge pin 620 and have recesses 621, 622 therein as shown. A magnet arrangement 623 may be employed to retain the second clamp arm 619 in clamping engagement with the clamp body 615 to support the instruments 520, 530 therein. Such arrangement enables the instruments 520, 530 to be quickly attached and detached to the input shaft 610. Other embodiments may employ threaded fasteners, clips, etc. to retain the second clamp arm 619 in clamping engagement with the clamp body 615.

It will be further appreciated that depending upon how the cables are attached to the tool docking assembly 600, movement of the handle portions of the surgical instruments 520, 530 causes the cable controlled guide tube to impart laparoscopic-like movement of the distal tips of the flexible portions of the surgical instruments. For example, when the handle is lifted up, the cable controlled working channel through which the flexible working portion extends may move the tip portion downward or upward depending upon how the cables are coupled to the tool docking stations. Likewise, when the handle is moved left, the working channel may cause the distal tip to move left or right. It will be further appreciated that the unique and novel features of the various embodiments of the flexible user interface support assembly 510 of the present invention enable the control cables for the cable controlled guide tube system to remain in any desired fixed position after the pivotal motions applied to the tool docking assembly or the surgical instruments docked therein have been discontinued.

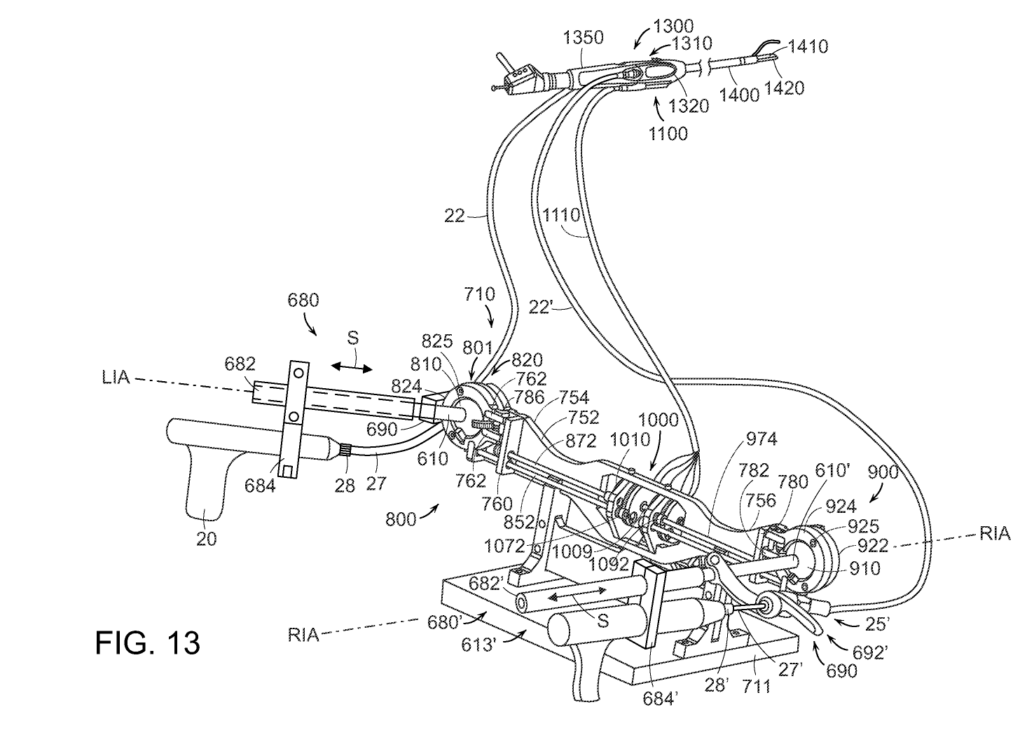

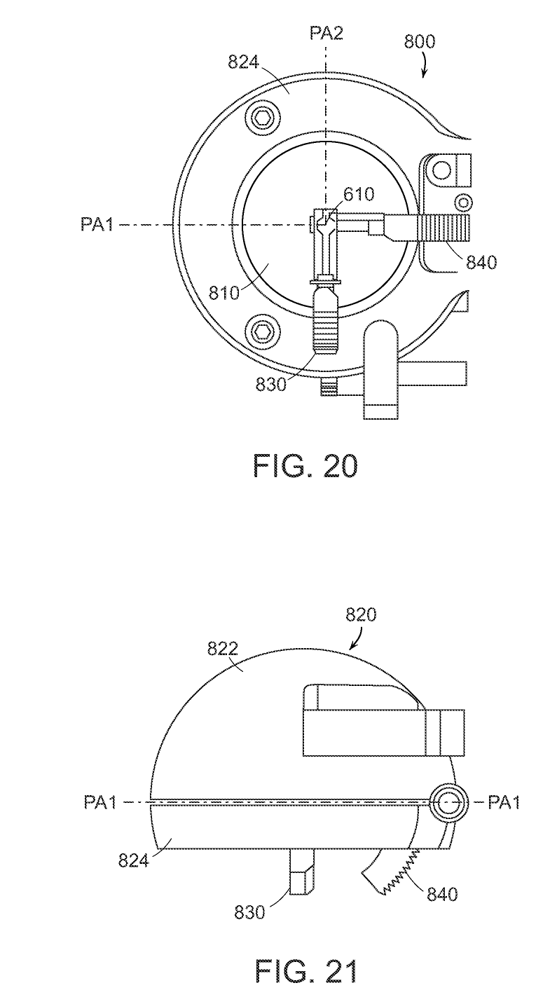

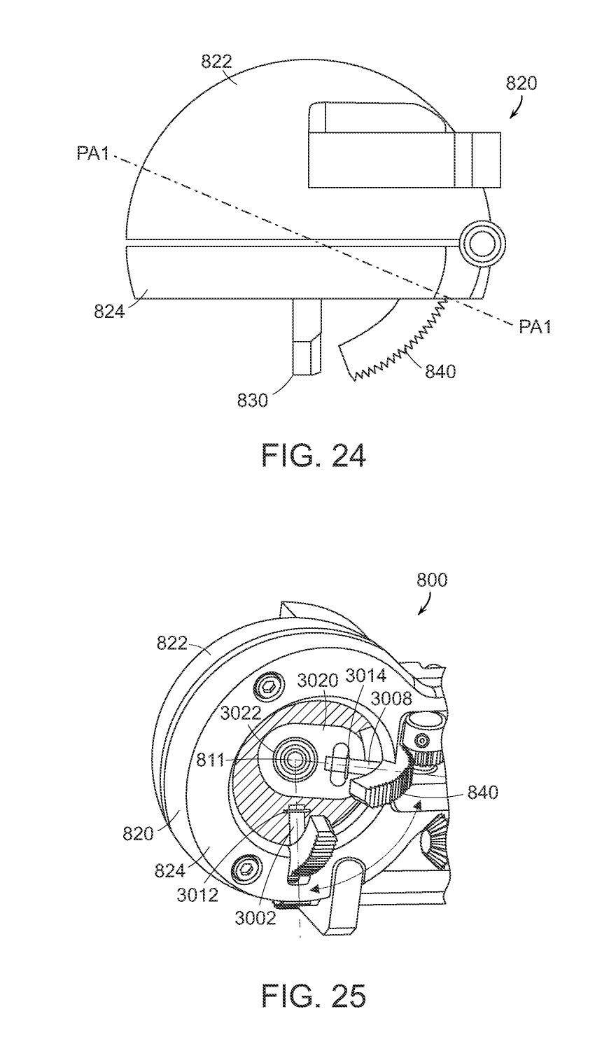

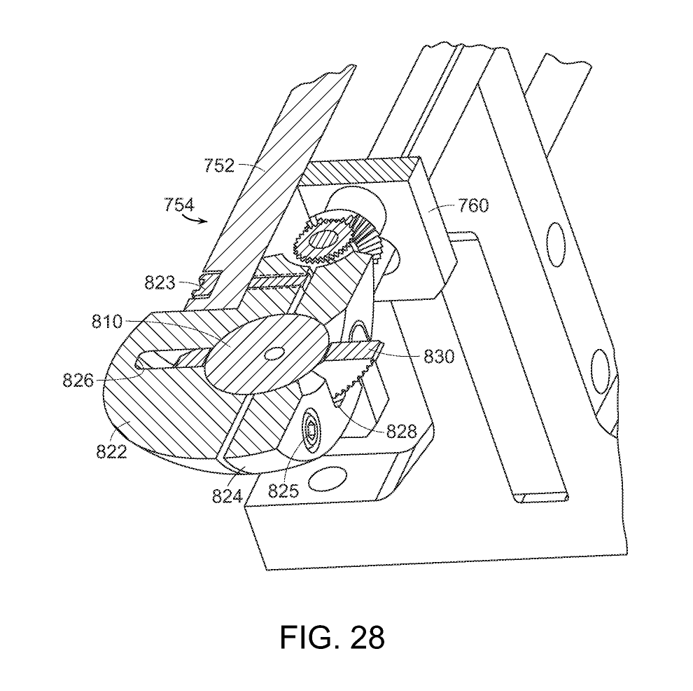

FIGS. 13-32 illustrate another flexible user interface support assembly embodiment generally designated as 710 that may operably support two or more conventional endoscopic surgical instruments 20, 20′ in connection with a cable-controlled, steerable guide tube assembly 1300. As can be seen in FIG. 13, an embodiment of the flexible interface support assembly 710 may include a surgical tool docking assembly 750 that may be operably attached to a support surface 711 such as, for example, a conventional work stand, a portion of a bed, etc. Various embodiments of the mounting assembly 750 may include a central cross bar 752 that may be clamped onto or otherwise fastened to the support surface 711 or, if desired, a conventional tool stand as was described hereinabove. The mounting assembly 750 may include a “first” or left tool docking station generally designated as 800 and a “second” or right tool docking station generally designated as 900. The left tool docking station 800 may be operably attached to a left end 754 of the central cross bar 752 and the right tool docking station 900 may be mounted to the right end 756 of the central cross bar 752.

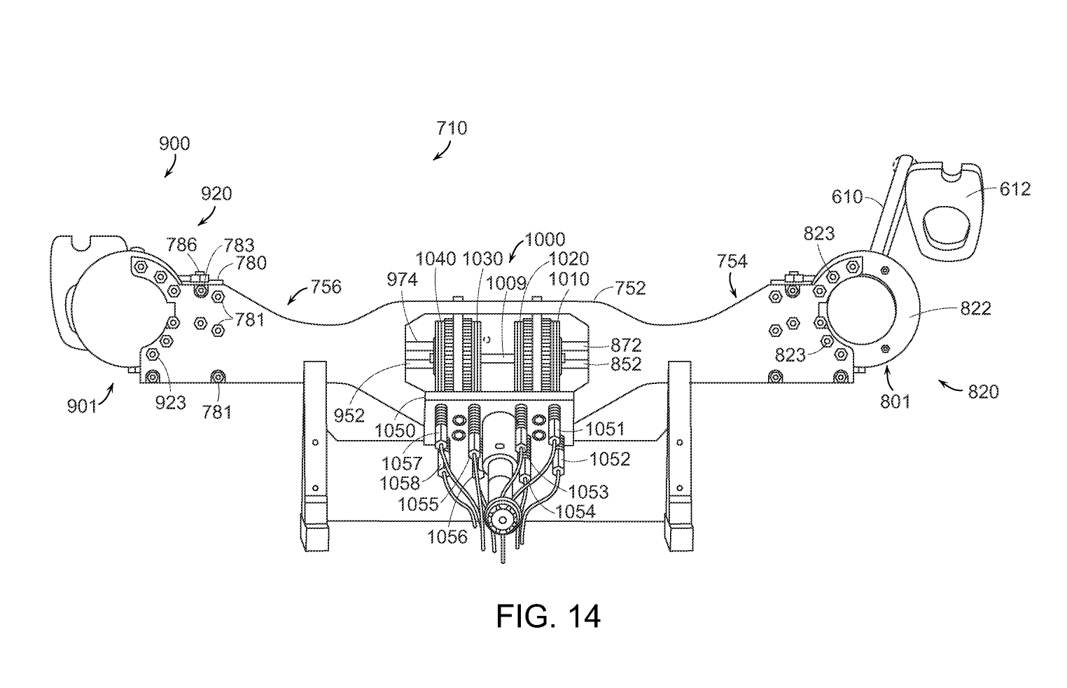

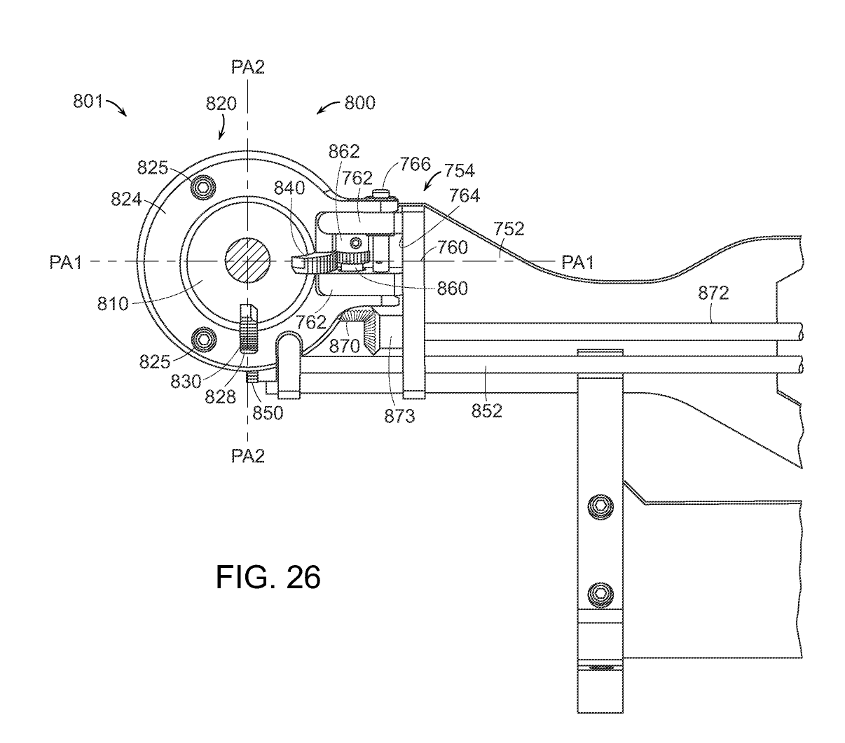

In various embodiments, the left tool docking station 800 may include a “first” or left ball and socket assembly 801. The left ball and socket assembly 801 may include a left sphere assembly 810 that is rotatably supported within a left housing assembly 820. Left housing assembly 820 may comprise, for example, a left sphere holder plate 822 that may be coupled to the left end 754 of the central cross bar 752 by, for example, screws 823 or other suitable fastener arrangements. See FIG. 14. The left housing assembly 820 may further include a left clamp plate 824 that is coupled to the left sphere holder plate 822 by screws 825 or other suitable fasteners. In addition, a left side plate 760 is attached to the left side 754 of the central cross bar 752. See FIGS. 13 and 26. A pair of spaced horizontal plates 762 are attached to the left side 754 of the central cross bar 752 by, for example, screws (not shown). The left clamp plate 824 may be configured to be journaled on a left hinge pin 766 that extends between the plates 762. The left clamp plate 24 allows the user to adjustably tension the sphere assembly 810 within a cavity 3000 formed by the left housing assembly 820 to generate a desired amount of resistance to for example retain the sphere assembly 810 and the surgical instrument 20 attached thereto in position when the clinician discontinues application of a positioning motion thereto. That is, when the clinician removes his or her hands from the surgical instrument 20, the friction created between the clamping plate 824 and the sphere assembly 810 will retain the sphere assembly and surgical instrument in that position. The left hinge pin 766 defines a first vertical axis FVA-FVA about which the first ball and socket assembly 801 may pivot relative to the central cross bar 752. See FIG. 26.

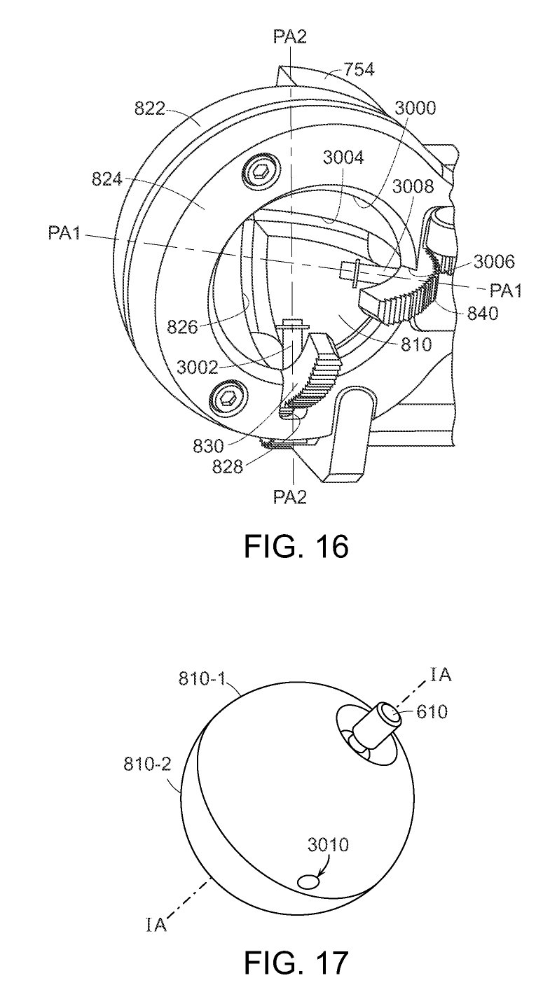

In various embodiments, the housing 820 acts as an unmovable reference or “ground' for the ball and socket system. Assembled within the spherical cavity 3000 is a vertical output gear segment 830 that has a primary axis of “PA1-PA1” that passes through the center of the spherical cavity 3000. This vertical output gear segment 830 may be constrained such that it can rotate about its horizontal primary axis of rotation PA1-PA1 by way of channels 826, 828 provided in the unmovable housing 820. By allowing the face of the gear segment 830 to ride on the walls of these channels 826, 828, the gear segment 830 is now unable to move in any plane other than which is normal to its axis of rotation PA1-PA1. See FIG. 16.

These embodiments may further include a sphere 810 that serves to “anchor” the axis rotation of the gear segment 830. In particular, a shaft 3002 extends from the gear segment 830 into the center of the sphere 810. In this manner, the gear segment 830, which was already constrained to motion in one plane can now be considered constrained to prevent translation in all directions and only allowing rotation about horizontal axis PA1-PA1 which passes through the center of the sphere 810. In addition, a user input shaft 610 may be attached to the sphere 810 for coupling surgical instruments or other articulatable user interfaces as will be discussed in further detail below. Movement of the input shaft 610 in any direction is translated into a proportional rotation of the gear segment 830 around horizontal input axis PA1-PA1 without regard for any input motion that occurs off axis. More specifically, an input motion by the user to the sphere 810 via the input shaft 610 will result in rotation of the gear segment 830 only if some element of the input is in the vertical direction. Thus, if the input motion were only in the horizontal direction, no relative motion would be registered on the vertical output gear 830.

As can also be seen in FIG. 16, the left tool docking station 800 further includes a horizontal input gear segment 840 which can be constrained in a similar manner to the vertical input gear segment 830 but with the horizontal input gear segment 840 oriented 90° relative thereto. The horizontal input gear segment 840 is oriented to rotate about a vertical axis PA2-PA2. The horizontal input gear segment 840 is constrained such that it is only able to rotate about its vertical primary axis of rotation PA2-PA2 by way of channels 3004, 3006 provided in the unmovable housing 820. By allowing the face of the gear segment 840 to ride on the walls of these channels 3004, 3006 the gear segment 840 is now unable to move in any plane other than which is normal to its axis of rotation PA2-PA2. A shaft 3008 extends from the gear segment 840 into the center of the sphere 810. In this manner, the gear segment 840 which was already constrained to motion in one plane can now be considered constrained to prevent translation in all directions and only allowing rotation about vertical axis PA2-PA2 which passes through the center of the sphere 810. Thus, the shafts 3002, 3008 extending respectively from gear segments 830, 840 towards the center of the sphere 810 are constrained to be 90° from the input shaft 610 wherein the surgical instruments or tools are mounted.

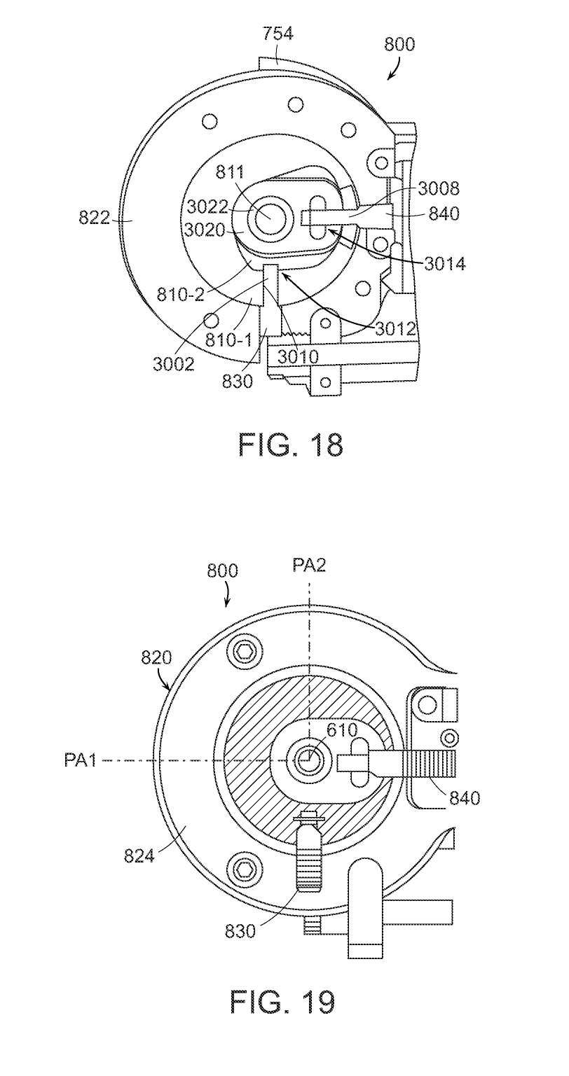

In preferred embodiments, it is desirable for the shafts 3002, 3008 to be round to facilitate rotation of the sphere 810 relative to the gear 830, 840 along the axis of the shaft 3002, 3008. It will be appreciated that in such embodiments, the angle defined by these two shafts 3002, 3008 is dynamic to enable the system to achieve the desired motions. As can be seen in FIGS. 17 and 18, the shaft on the gear segment 830 is constrained to the plane originally described by the two axes normal to the input shaft 610 by extending through a hole 3010 in the sphere 810. Shaft 3002 rotatably extends through the hole 2010 and is retained in position by an e-clip 3012. A central rotator 3020 is movably supported within the sphere 810. The shaft 3008 of the gear segment 840 is affixed to the central rotator 3020 by an e-clip 3014. The input shaft is attached to a bearing 3022 within the central rotator 3020 by an input shaft screw 611 such that the central rotator 3020 can freely rotate about the input axis IA-IA defined by input shaft 610 but is constrained to the same plane as that of the shafts 3002, 3008 of the two gear segments 830, 840. To aid in the fabrication process, the sphere 810 may comprise a front component 810-1 and a rear component 810-2 that is attached thereto.

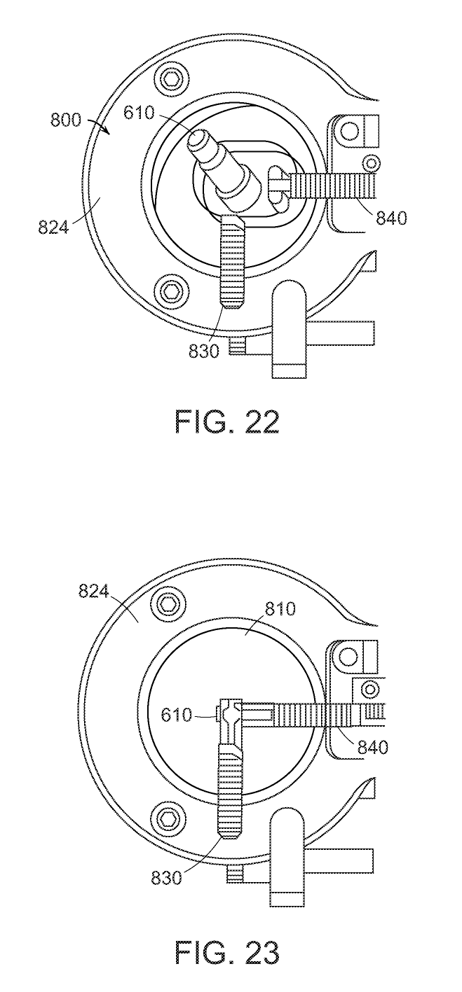

FIGS. 19-21 depict various components when the input shaft is in a central or “neutral” position. As can be seen in those Figures, the two gear segments 830, 840 are at right angles to each other. Once the input shaft 610 has been moved to a non-neutral position, the axes of the shafts 830, 840 have also moved to an alternate position (although the faces of the gears 830, 840 are still constrained to the slots (826, 828 for gear segment 830 and 3004, 3006 for gear segment 840). See FIGS. 22-24. Furthermore, by sighting down the input shaft 610 with the sphere 810 in a non-neutral position, it can be seen that the orientation of the two gear shafts 3002, 3008 relative to each other has changed. See FIG. 25.

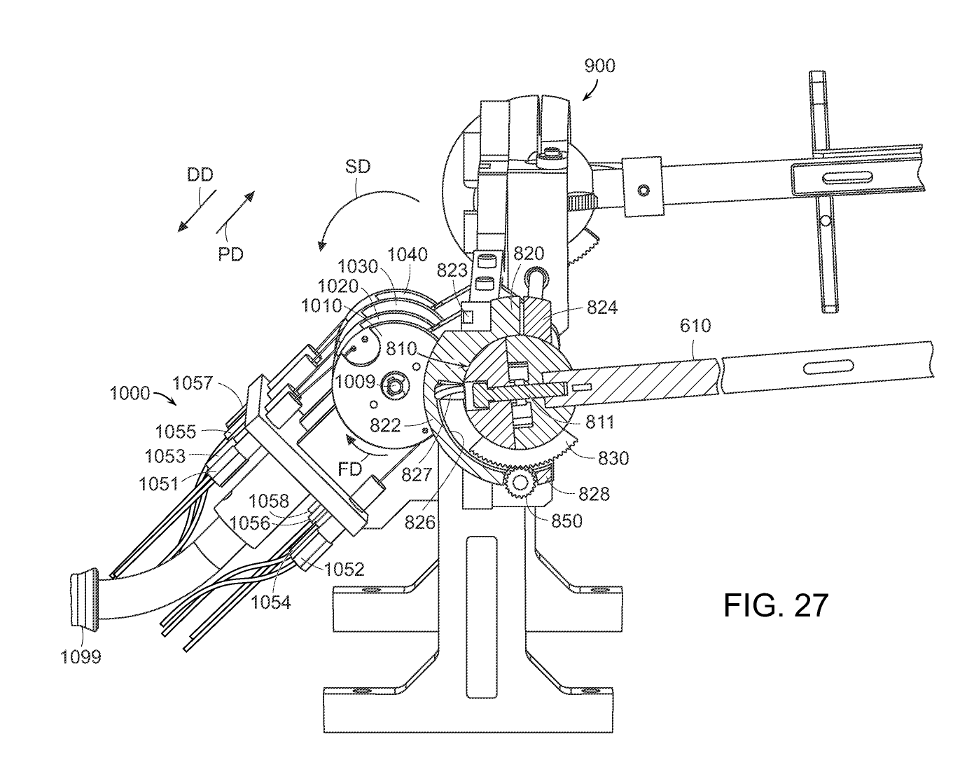

As can be seen in FIG. 13, the endoscopic surgical instrument 20 may be attached to the left tool docking station 800 by a unique and novel tool mounting assembly generally designated as 680 that comprises a left tool mounting tube 682 that is slidably received on the left input shaft 610. A tool clamp assembly 684 is clamped onto or otherwise attached to the left tool mounting tube 682 and is configured to releasably clamp or otherwise engage the surgical instrument 20. The left input shaft 610 may be attached to the left sphere assembly 810 by a screw 811. See FIG. 27. In general, the left input shaft 610, the left tool mounting tube 682 and the tool clamp assembly 684 may be collectively referred to herein as the left tool mounting assembly 613.

In various embodiments, the first left driver gear 830 is positioned in meshing engagement with a first vertical pinion gear 850 that is attached to a first left drive shaft 852. Thus, rotation of the left sphere assembly 810 about the horizontal pivot axis PA1-PA1 will cause the first left driver gear 830 and first vertical pinion gear 850 to impart a rotary motion of a first left drive shaft 852. Similarly, the second left driver gear 840 is in meshing engagement with a first horizontal pinion gear 862 that is attached to a left pinion shaft 860 mounted between the plates 762. Thus, rotation of the left ball and socket assembly 801 about the vertical pivot axis FVA-FVA will cause the second left driver gear 840 and first horizontal pinion 860 to impart a rotary motion to the left pinion shaft 862 and a first left miter gear 870 attached thereto. The first left miter gear 870 is in meshing engagement with a second left miter gear 873 that is attached to a second left drive shaft 872. The first and second left drive shafts 852, 872, respectively, may extend through the left side plate 760 and be rotatably supported therein in corresponding bearings (not shown). The first and second left drive shafts 852, 872 serve to impart rotary drive motions to a centrally disposed cable drive assembly 1000 as will be discussed in further detail below.

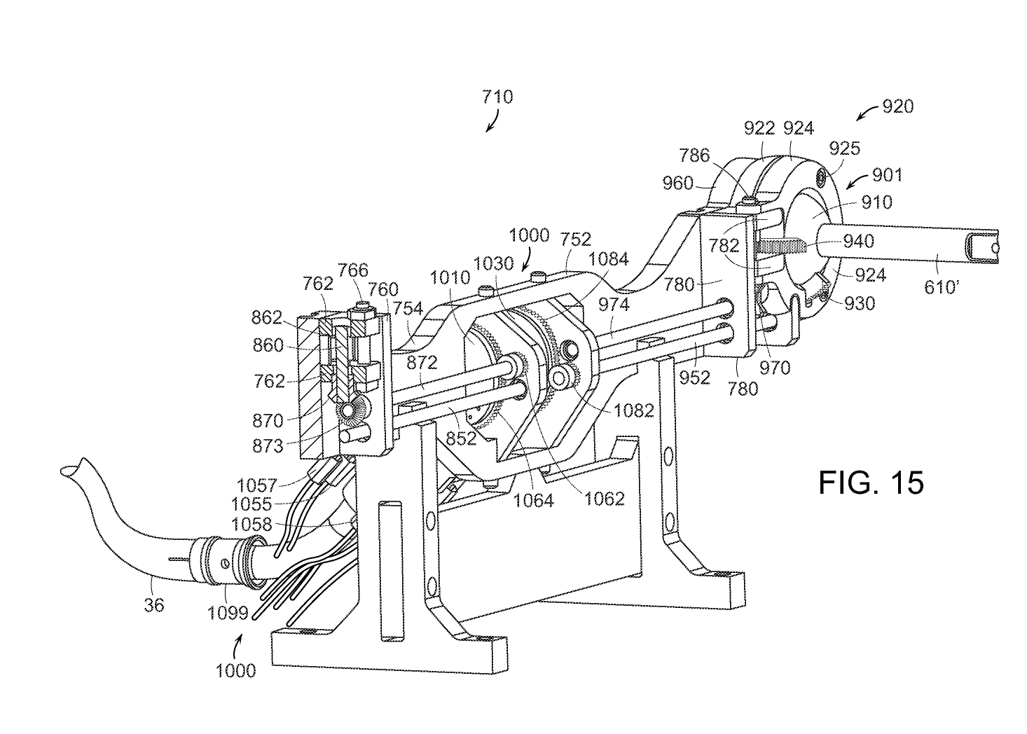

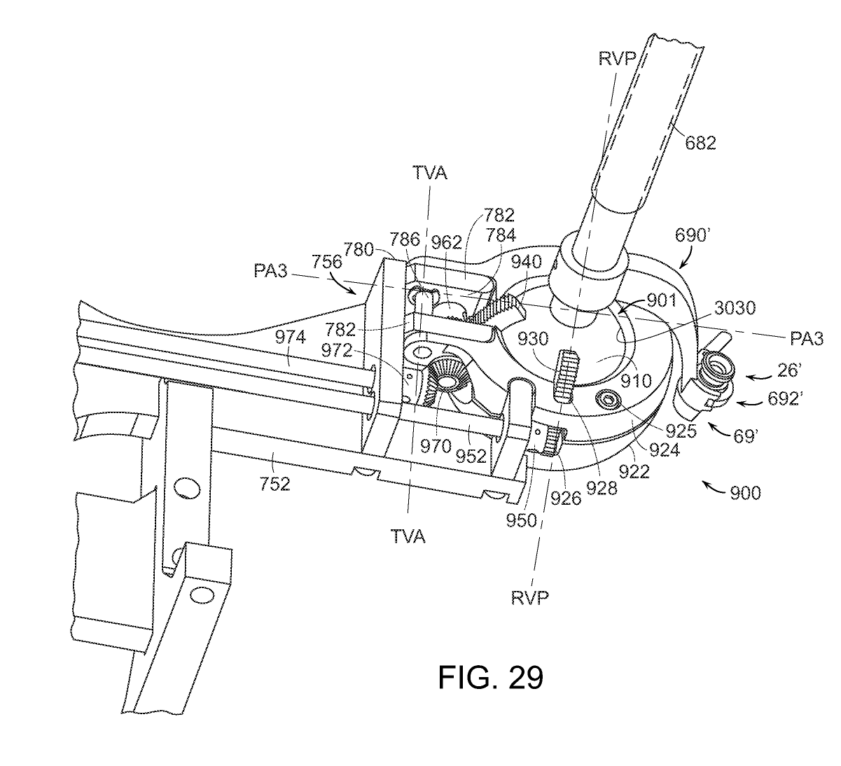

The mounting assembly 750 may also include a “second” or right tool docking station 900 that is mounted to the right end 756 of the central cross bar 752 and is substantially identical in construction and operation as the left tool docking station 800. See FIG. 13. For example, in various embodiments, the right tool docking station 900 includes a “second” or “right” ball and socket assembly 901. The right ball and socket assembly 901 may include a right sphere assembly 910 that is rotatably supported within a right housing assembly 920. Right housing assembly 920 may comprise, for example, a right sphere holder plate 922 that may be coupled to the right end 756 of the central cross bar 752 by, for example, screws 923 or other suitable fastener arrangements. See FIG. 15. The right housing assembly 920 may further include a right clamp plate 924 that is coupled to the right sphere holder plate 922 by screws 925 or other suitable fasteners. In addition, a right side plate 780 is attached to the right side 756 of the central cross bar 752 by screws 781 or other suitable fasteners. See FIGS. 14 and 15. A pair of spaced horizontal plates 782 are attached to the right side plate 760 by, for example, screws 783. The right clamp plate 924 may be configured to be journaled on a right hinge pin 786 extending between the plates 782. The right clamp plate 924 allows the user to adjustably tension on the sphere 910 within the right housing assembly 920 to generate a desired amount of resistance to, for example, retain the sphere 910 and surgical instrument 20′ attached thereto in position when the clinician discontinues the application of actuation motion thereto. That is, when the clinician removes his or her hands from the surgical instrument 20′, the friction created between the clamping plate 924 and the sphere assembly 910 will retain the sphere assembly and surgical instrument in that position. The right hinge pin 786 permits the second ball and socket assembly 901 to pivot about vertical axis TVA-TVA. See FIG. 29.

In various embodiments, the right housing assembly 920 acts as an unmovable reference or “ground’ for the right tool docking station 900. Within this unmovable reference 920 is a spherical cavity 3030 which supports the sphere assembly 910 and gear segments 930 and 940. The vertical output gear segment 930 has a shaft (not shown) and is mounted in the above-described manner such that its horizontal primary axis of rotation “PA3-PA3” passes through the center of the spherical cavity 3030. This vertical output gear segment 930 can then be constrained such that it is only able to rotate about its horizontal primary axis of rotation PA3-PA3 by way of channels 926, 928 provided in the unmovable right housing assembly 920. By allowing the face of the gear segment 930 to ride on the walls of these channels 926, 928, the gear segment 930 is now unable to move in any plane other than which is normal to its axis of rotation PA3-PA3. See FIG. 29.

As can also be seen in FIG. 29, the right tool docking station 900 further includes a horizontal input gear segment 940 which can be constrained in a similar manner to the vertical input gear segment 930 but with the horizontal input gear segment 940 oriented 90° relative thereto. The horizontal input gear segment 940 is oriented to rotate about a vertical axis RVP-RVP that also passes through the spherical cavity 3030. The horizontal input gear segment 940 has a shaft (not shown) and is constrained in the above-described manner such that it is only able to rotate about its vertical primary axis of rotation PA4-PA4 by way of channels (not shown) provided in the unmovable right housing assembly 920. As was discussed above, such arrangement constrains the horizontal gear input segment 940 such that it is unable to move in any plane other than which is normal to its axis of rotation PA4-PA4.

As can be seen in FIG. 13, the endoscopic surgical instrument 20 may be attached to the right tool docking station 900 by a unique and novel tool mounting assembly generally designated as 680′ that comprises a right tool mounting tube 682′ that is slidably received on the right input shaft 610′. A right tool clamp assembly 684′ is clamped onto or otherwise attached to the right tool mounting tube 682′ and is configured to releasably clamp or otherwise engage the surgical instrument 20′. The right input shaft 610′ may be attached to the right sphere assembly 910 by a screw (not shown). In general, the right input shaft 610′, the right tool mounting tube 682′ and the right tool clamp assembly 684′ may be collectively referred to herein as the right tool mounting assembly 613′.

In various embodiments, the first right drive gear segment 930 is positioned in meshing engagement with a right vertical pinion gear 950 that is attached to a first right drive shaft 952. Thus, rotation of the right sphere 910 about the primary horizontal pivot axis PA4-PA4 will cause the third driver gear 930 and third vertical pinion gear 950 to impart a rotary motion to a first right drive shaft 952. Similarly, the fourth driver gear 940 is in meshing engagement with a fourth horizontal pinion gear 962 that is attached to a pinion shaft 960 mounted between the plates 962. Thus, rotation of the second ball and socket assembly 901 about the primary vertical pivot axis PA4-PA4 will cause the fourth driver gear 940 and fourth horizontal pinion 960 to impart a rotary motion to the pinion shaft 962 and a first right miter gear 970. The first right miter gear 970 is in meshing engagement with a second right miter gear 972 that is attached to a second right drive shaft 974.

As was mentioned above, the endoscopic surgical instrument 20 may be attached to the left tool docking station 800 by a unique and novel tool mounting assembly generally designated as 680 that comprises a left tool mounting tube 682 that is slidably received on the input shaft 610. A tool clamp assembly 684 is clamped onto or otherwise attached to the left tool mounting tube 682 and is configured to releasably clamp or otherwise engage the surgical instrument 20. See FIG. 13. In other embodiments, a clamp 614 of the type described above, may also be successfully employed to couple the surgical instrument 20 to the left tool mounting tube 682.

Similarly, the endoscopic surgical instrument 20′ may be attached to the right tool docking station 900 by a unique and novel tool mounting assembly generally designated as 680′ that comprises a left tool mounting tube 682′ that is slidably received on the input shaft 610′. A tool clamp assembly 684′ is clamped onto or otherwise attached to the left tool mounting tube 682′ and is configured to releasably clamp or otherwise engage the surgical instrument 20′. See FIG. 13. In other embodiments, a clamp 614 of the type described above, may also be successfully employed to couple the surgical instrument 20′ to the right tool mounting tube 682′.

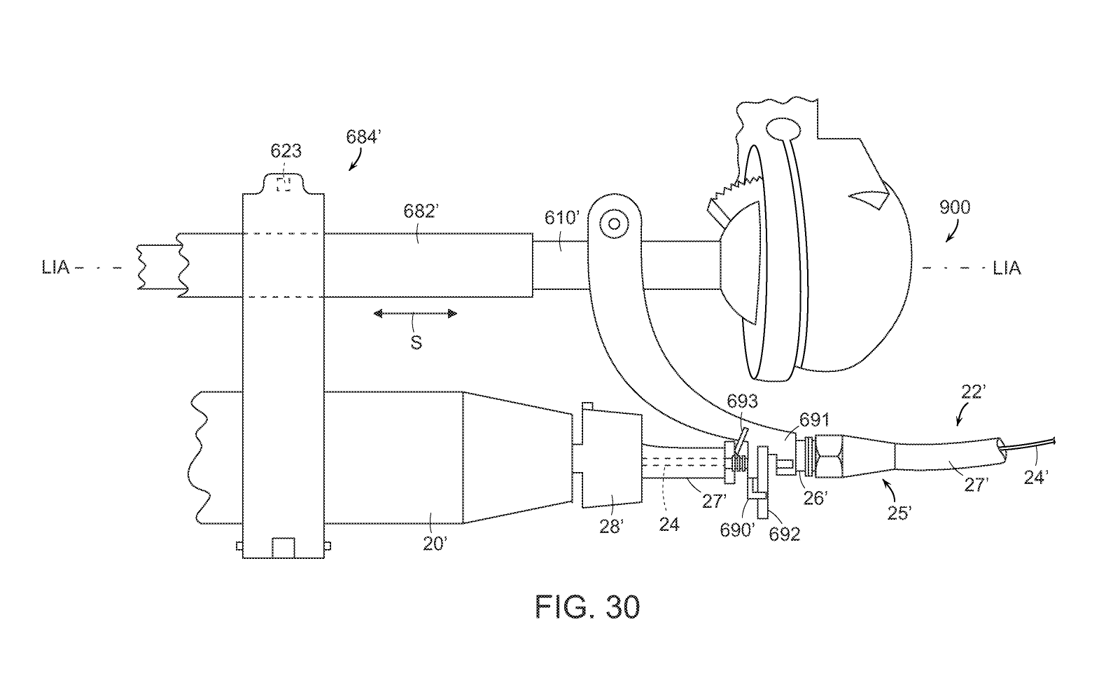

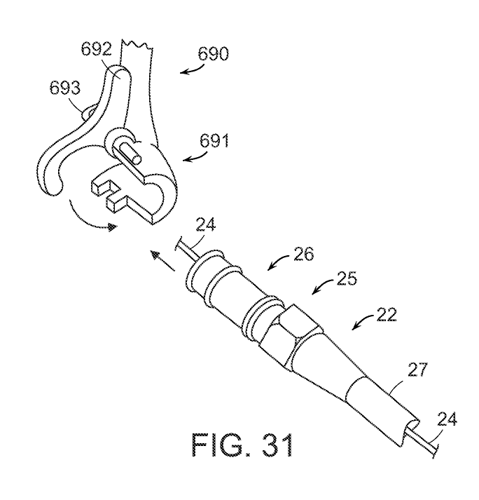

As can be seen in FIG. 13, various embodiments of the present invention may also employ cable mounting assemblies generally designated as 690, 690′ for respectively supporting the flexible working portions 22, 22′ of the surgical instruments 20, 20′. A cable mounting assembly 690 may include a ferrule coupling portion 691 that includes a movable latch 692 that is movable between a latched and unlatched position. See FIGS. 30 and 31. A spring 693 may be employed to bias the latch 692 into the latched position. The flexible working portion 22 may comprise a hollow outer sheath 27 through which an operating cable 24 from the surgical instrument 20 movably extends. The flexible working portion 22 may further have a ferrule portion 25 that has a flanged barrel 26 that is sized to be received within the ferrule coupling portion 691. When the flanged barrel 26 has been inserted into the ferrule coupling portion 691, it can be retained therein when the latch 692 is moved to the latched position. Likewise, as can be seen in FIG. 31, the flexible working portion 22′ may comprise a hollow outer sheath 23′ through which an operating cable 24′ from the surgical instrument 20′ movably extends. The flexible working portion 22′ further has a ferrule portion 25′ that has a flanged barrel 26′ that is sized to be received within the ferrule coupling portion 691. When the flanged barrel 26′ has been inserted into the ferrule coupling portion 691, it can be retained therein when the latch 692 is moved to the latched position.

As described above, the tool mounting assembly 680 will enable the clinician to move the surgical instrument 20 on the input shaft 610 along the left input axis LIA-LIA in the directions represented by arrow “S” in FIG. 13. Thus, such movement of the surgical instrument 20 will cause the flexible cable 24 protruding therefrom to move in and out of the sheath 22 which will cause the instrument tip (not shown) to move in and out of a cable docking station 1100 which will be described in further detail below. Likewise, the tool mounting assembly 680′ will enable the clinician to move the surgical instrument 20′ on the input shaft 610′ along a right tool axis “RIA-RIA” in the directions represented by arrow “S” in FIG. 13. Thus, such movement of the surgical instrument 20′ will cause the flexible cable 24′ protruding therefrom to move in and out of the sheath 22′ which will cause the instrument tip (not shown) to move in and out of a cable docking station 1100.

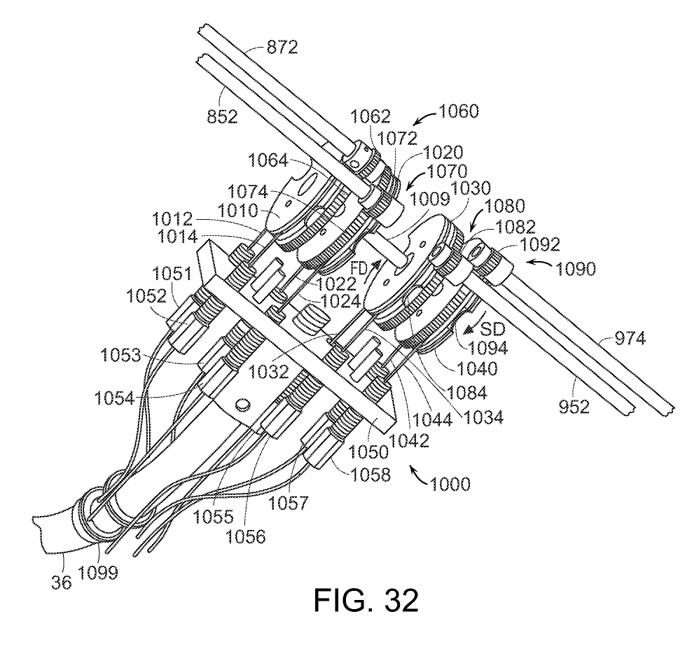

As can be seen in FIG. 13, the first left drive shaft 852, the second left drive shaft 872, the first right drive shaft 952 and the second right drive shaft 974 are configured to drivingly interface with a cable drive assembly 1000 that is centrally disposed on the cross bar 752. In various embodiments, the cable drive assembly 1000 may include a first cable pulley 1010, a second cable pulley 1020, a third cable pulley 1030, and a fourth cable pulley 1040 that are journaled an axle 1009 for controlling cables in connection with a cable-controlled steerable guide tube assembly 1300 or other cable-controlled steerable guide tube assemblies such as the steerable guide tube assembly 200 as described above. See FIG. 32.

The first cable pulley 1010 has a first upper cable 1012 and a first lower cable 1014 attached thereto. The first upper and lower cables 1012, 1014 are attached to the first cable pulley such that rotation of the first cable pulley 1010 in first direction “FD” (FIGS. 27 and 32) causes the first upper cable 1012 to be pulled in a proximal direction “PD” and the first lower cable 1014 to be pushed in a distal direction “DD”. Likewise, rotation of the first cable pulley 1010 in a second direction “SD” causes the first upper cable 102 to be pushed in the distal direction “DD” and the first lower cable 1014 to be pulled in a proximal direction “PD”. The first upper and lower cables 1012, 1014 extend through corresponding hex coil pipe adjuster assemblies 1051, 1052, respectively mounted to a mounting plate 1050 and are ultimately coupled to the steerable guide tube assembly 1300 as will be discussed in further detail below. As can be seen in FIG. 32, rotation of the first cable pulley 1010 is controlled by the second left drive shaft 872 that is coupled to a drive gear train 1060 that consists of intermeshing gears 1062, 1064. Gear 1062 is attached to the second left drive shaft 872. Gear 1064 is attached to the first cable pulley 1010 for rotational travel therewith about an axle 1061. Thus, rotation of the second left drive shaft 872 will cause the gears 1062, 1064 to rotate and ultimately cause the first cable pulley 1010 to rotate as well.

Likewise, the second cable pulley 1020 has a second upper cable 1022 and a second lower cable 1024 attached thereto. The second upper and lower cables 1022, 1024 are attached to the second cable pulley 1020 such that rotation of the second cable pulley 1020 in first direction “FD” causes the second upper cable 1022 to be pulled in a proximal direction and the second lower cable to be pushed in a distal direction. The second upper and lower cables 1022, 1024 extend through corresponding hex coil pipe adjuster assemblies 1053, 1054, respectively in the mounting plate 1050 and are ultimately coupled to the steerable guide tube assembly 1300. Rotation of the second cable pulley 1020 is controlled by rotation of the first left drive shaft 852 that is coupled to a drive gear train 1070 that consists of intermeshing gears 1072, 1074. As can be seen in FIG. 32, the gear 1072 is attached to the first left drive shaft 852. Gear 1074 is attached to the second cable pulley 1014 for rotational travel therewith. Thus, rotation of the first left drive shaft 852 will cause gears 1072, 1074 and ultimately the cable pulley 1014 to rotate.

The third cable pulley 1030 has a third upper cable 1032 and a third lower cable 1034 attached thereto. The third upper and lower cables 1032, 1034 are attached to the third cable pulley 1030 such that rotation of the third cable pulley 1030 in the first direction “FD” causes the third upper cable 1032 to be pulled in the proximal direction and the third lower cable 1034 to be pushed in the distal direction. The third upper and lower cables 1032, 1034 extend through corresponding hex coil pipe adjuster assemblies 1055, 1056, respectively attached to mounting plate 1050 and are ultimately coupled to the steerable guide tube assembly 1300. Rotation of the third cable pulley 1030 is controlled by rotation of the first right drive shaft 952 that is coupled to a drive gear train 1080 that consists of intermeshing gears 1082, 1084. As can be seen in FIG. 32, the gear 1082 is attached to the first right drive shaft 952. Gear 1084 is attached to the third cable pullet 1030 for rotational travel therewith. Rotation of the first right drive shaft 952 will cause gears 1082, 1084 and ultimately, the third cable pulley 1030 to rotate.

The fourth cable pulley 1040 has a fourth upper cable 1042 and a fourth lower cable 1044 attached thereto. The fourth upper and lower cables 1042, 1044 are attached to the fourth cable pulley 1040 such that rotation of the fourth cable pulley 1040 in first direction “FD” causes the fourth upper cable 1042 to be pulled in the proximal direction and the fourth lower cable 1044 to be pushed in the distal direction. The fourth upper and lower cables 1042, 1044 extend through corresponding hex coil pipe adjuster assemblies 1057, 1058 in the mounting plate 1050 and are ultimately coupled to the steerable guide tube assembly 1300. Rotation of the fourth cable pulley 1040 is controlled by rotation of the second right drive shaft 974 that is coupled to a drive gear train 1090 that consists of intermeshing gears 1092, 1094. As can be seen in FIG. 32, the gear 1092 is attached to the second right drive shaft 974. Gear 1094 is attached to the fourth cable pulley 1040 for rotational travel therewith. Thus, rotation of the second right drive shaft 974 will cause gears 1092, 1094 and ultimately the fourth cable pulley 1040 to rotate.

In various embodiments of the present invention, the cables 1012, 1014, 1022, 1024, 1032, 1034, 1042, 1044 are configured to be operably coupled to a steerable guide tube assembly 1300 by a unique and novel cable docking station 1100. In various embodiments, for example, the cable docking station 1100 is clamped or otherwise attached to a flexible gooseneck mounting tube 36 that is attached to a mounting collar 1099 that is affixed to the cable drive assembly 1000. The cables 1012, 1014, 1022, 1024, 1032, 1034, 1042, and 1044 may extend into a hollow sheath 1110 that attaches to the cable docking station 1100. See FIGS. 13, 33, and 34.

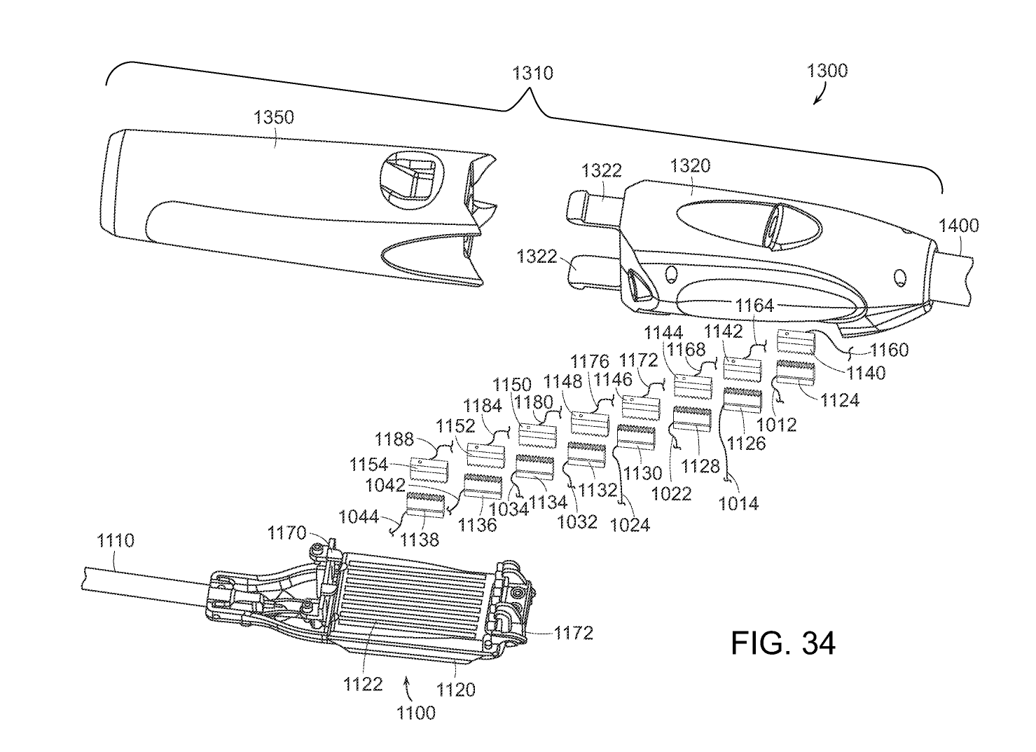

As can be seen in FIG. 34, the cable docking station 1100 may include a bottom plate 1120 that operably supports a clamp plate 1122. Clamp plate 1122 may support a series of proximal cable couplers in the form of lower pitch racks that are attached to the distal ends of cables 1012, 1014, 1022, 1024, 1032, 1034, 1042, 1044. For example, a lower pitch rack 1124 may be attached to the distal end of cable 1012. Lower pitch rack 1226 may be attached to the distal end of cable 1014. Lower pitch rack 1228 may be attached to the distal end of cable 1022. Lower pitch rack 1130 is attached to the distal end of cable 1024. Lower pitch rack 1132 is attached to the distal end of cable 1032. Lower pitch rack 1134 may be attached to the distal end of cable 1034. Lower pitch rack 1136 may be attached to the distal end of cable 1042. Lower pitch rack 1138 may be attached to the distal end of cable 1044. Lower pitch racks 1124, 1126, 1128, 1130, 1132, 1134, 1136, and 1138 may be configured to mesh with corresponding distal cable couplers in the form of upper pitch racks 1140, 1142, 1144, 1146, 1148, 1150, 1152, 1154, respectively, that may be supported in the steerable guide tube assembly 1300.

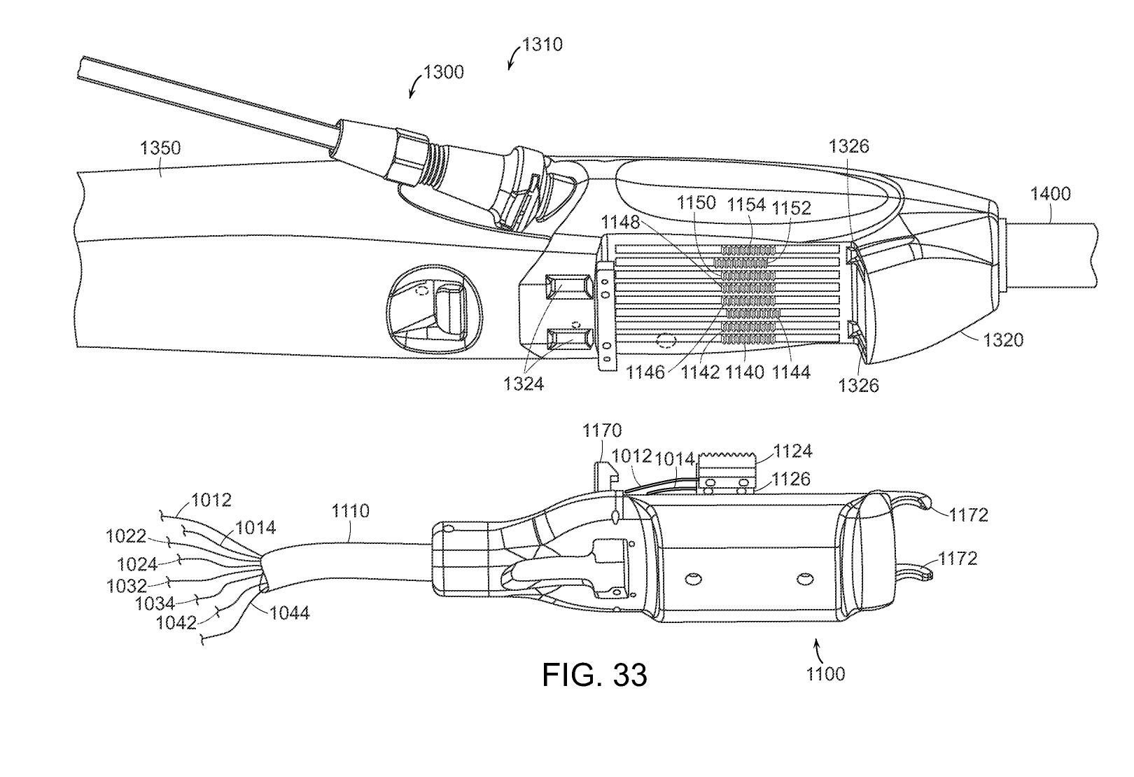

The steerable guide tube assembly 1300 may include a handle housing 1310 that may comprise a distal portion 1320 and a proximal portion 1350 that may be attached together by, for example, snap features 1322 on the distal portion 1320. As can be seen in FIG. 33, the distal housing portion 1320 may be formed with latch cavities 1324, 1326 that are adapted to be retainingly engaged by latch features 1170, 1172, respectively, that are operably attached to or otherwise formed on the bottom plate 1120 of the cable docking station 1100.

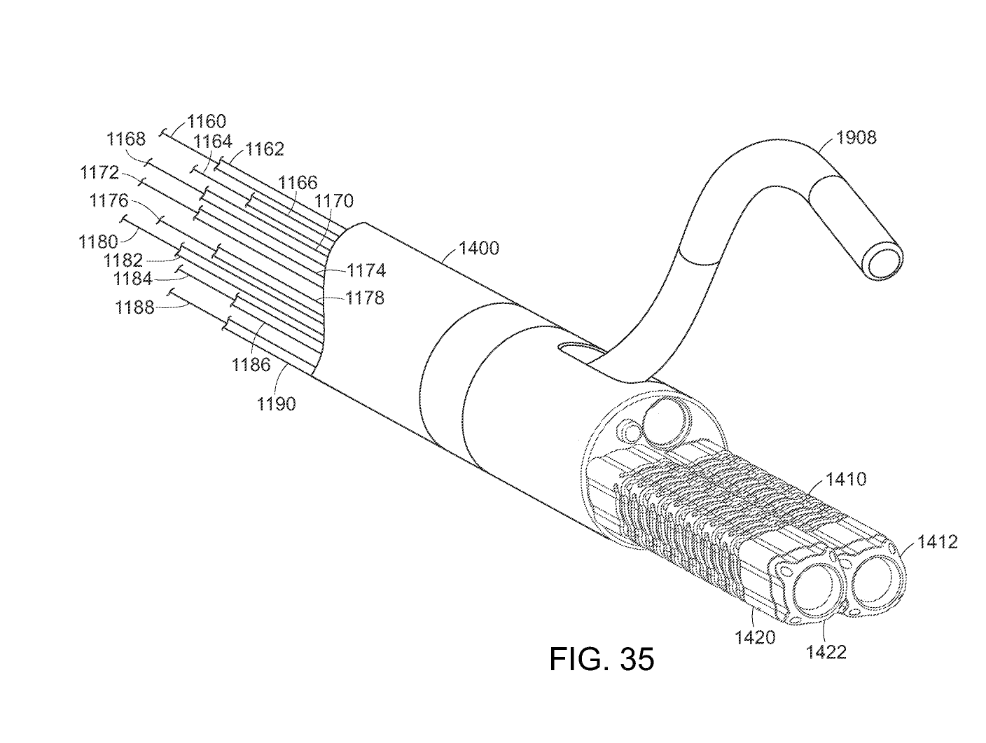

The steerable guide tube assembly 1300 may include a flexible insertion tube 1400 that operably supports two or more steerable working channels 1410 and 1420. For example, when the handle housing 1310 is docked to the cable docking station 1100, the distal end portion 1412 of the left working channel 1410 may be steered by manipulating cables 1012, 1014, 1022 and 1024 and the distal end portion 1142 of the working channel 1140 may be steered by cables 1032, 1034, 1042, 1044 as will be explained in further detail below. In particular, in various embodiments, the upper pitch racks 1140, 1142, 1144, 1146, 1148, 1150, 1152, 1154 each have a distal cable segment attached thereto that extend through corresponding coil pipe segments supported in the flexible insertion tube 1400 to be coupled to the distal end portions 1412, 1422 of the steerable working channels 1410, 1420, respectively. See FIG. 35.

Referring to FIGS. 33 and 34, the upper pitch rack 1140 may be attached to a proximal end of a distal cable segment 1160 that extends through a coil pipe segment 1162 to be attached to the distal end portion 1412. The upper pitch rack 1142 may be attached to a proximal end of a distal cable segment 1164 that extends through a coil pipe segment 1166 to be attached to the distal end portion 1412. The upper pitch rack 1144 may be attached to a proximal end of a distal cable segment 1168 that extends through a coil pipe segment 1170 to be attached to the distal end portion 1412. The upper pitch rack 1146 may be attached to a proximal end of a distal cable segment 1172 that extends through a coil pipe segment 1174 to be attached to the distal end portion 1412.

Similarly, the upper pitch rack 1148 may be attached to a proximal end of a distal cable segment 1176 that extends through a coil pipe segment 1178 to be attached to the distal end portion 1422. The upper pitch rack 1150 may be attached to a proximal end of a distal cable segment 1180 that extends through a coil pipe segment 1182 to be attached to the distal end portion 1422. The upper pitch rack 1152 may be attached to a proximal end of a distal cable segment 1184 that extends through a coil pipe segment 1186 to be attached to the distal end portion 1422. The upper pitch rack 1154 may be attached to a proximal end of a distal cable segment 1188 that extends through a coil pipe segment 1190 to be attached to the distal end portion 1422.

Thus, the flexible user interface support assembly 710 may be used as follows. Initially, the clinician may mount the endoscopic surgical instruments 20, 20′ to the corresponding tool mounting plate 612. As indicated above, the endoscopic surgical instruments 20, 20′ may comprise, for example endoscopes, lights, insufflation devices, cleaning devices, suction devices, hole-forming devices, imaging devices, cameras, graspers, clip appliers, loops, Radio Frequency (RF) ablation devices, harmonic ablation devices, scissors, knives, suturing devices, etc., a portion of which may operably extend through one of the working channels 1410, 1420 in the steerable guide tube assembly 1300. The steerable guide tube assembly 1300 may be “dockingly engaged with” the cable docking station 1100 by engaging the latches 1170, 1172 on the cable docking station 1100 with the respective latch cavities 1324, 1326 in the distal housing section 1320. Those of ordinary skill in the art will understand that the tool mounting plates 612 may be especially configured to mountingly interface with the type of endoscopic surgical instruments to be used. Once the endoscopic surgical instruments 20, 20′ are mounted to the user interface support assembly 710 and the steerable guide tube assembly 1300 has been docked on the cable docking station 1100, the flexible working portions 22, 22′ of the endoscopic surgical instruments 20, 20′ may be inserted through ports in the handle housing 1310 of the steerable guide tube assembly 1300 and out through the working channels 1410, 1420. The insertion tube portion 1400 may then be inserted into the patient, if it had not been previously inserted therein prior to installing the endoscopic surgical instruments 20, 20′.

When the steerable guide tube assembly 1300 has been docked onto the cable docking station 1100, cables 1012, 1014, 1022, 1024 are coupled to their corresponding distal cable segments 1160, 1164, 1168, 1172 by virtue of the meshing engagement between the lower pitch racks 1124, 1126, 1128, 1130 with the respective corresponding upper pitch racks 1140, 1142, 1144, 1146. Similarly, cables 1032, 1034, 1042, and 1044 are coupled to their corresponding distal cable segments 1176, 1180, 1184, and 1188 by virtue of the meshing engagement between the lower pitch racks 1132, 1134, 1136, and 1138 with respective corresponding upper pitch racks 1148, 1150, 1152, and 1154. To manipulate the distal end portion 1412 of the working channel 1410 and thus the working portion 22 of the endoscopic tool 20 in the left and right direction, the clinician simply moves the endoscopic surgical instrument 20 in the direction in which he or she desires the end portion 1412 of the to flexible working channel 1410 to go and the coupled cables 1012, 1014, 1022, 1024, 1160, 1164, 1168, 1172 manipulate the distal end portion 1422 of the working channel 1420.

In various applications, the working channels may communicate with insufflation pressure in the abdomen. To maintain the desired pressure, commercially available seals 28, 28′ to prevent the insufflation pressure from leaking out through the flexible working portions 22, 22′. See FIG. 30. Seals 28, 28′, such as those manufactured by Ethicon-Endo Surgery, Inc. of Cincinnati, Ohio are couplable to the distal ends of the surgical instruments 20, 20′ and facilitate insertion of the operating cable or flexible portion 24, 24′ therethrough while maintaining an airtight seal with the outer sheath 27, 27′ of the corresponding flexible working portion 22, 22′.

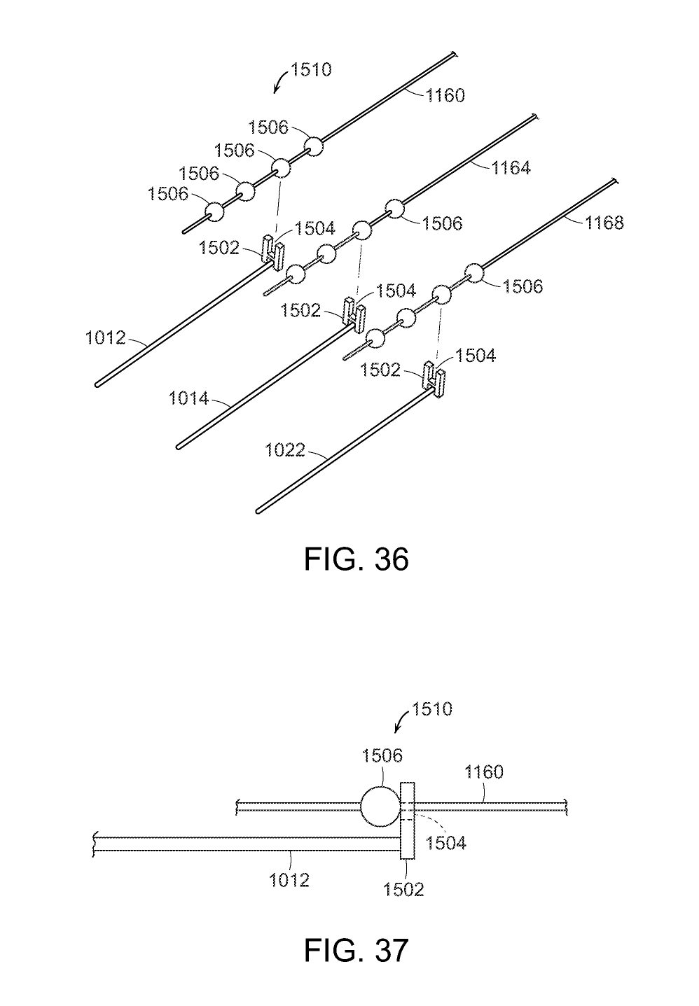

FIGS. 36 and 37 illustrate alternative cable coupling arrangement 1510 that may be effectively employed in the cable docking station 1100. For example, in this embodiment, the distal ends of cables 1012, 1014, 1022, 1024, 1032, 1034, 1042, 1044 would each have a proximal cable coupler in the form of a retention member 1502 attached thereto. Each retention member 1502 would have a groove 1504 therein sized to snappingly receive the proximal end of a corresponding distal cable segment 1160, 1164, 1168, 1172, 1176, 1180, 1184, 1188 therein. Each distal end of distal cable segments 1160, 1164, 1168, 1172, 1176, 1180, 1184, 1188 would have a distal cable coupler attached thereto in the form of at least one retention bead 1506 such that when the cable docking station 1100 is attached to the steerable guide tube assembly 1300, the cable segments cable segments 1160, 1164, 1168, 1172, 1176, 1180, 1184, 1188 snap into the groove 1504 in the corresponding retention member 1502 attached to cables 1012, 1014, 1022, 1024, 1032, 1034, 1042, 1044 and the retention beads 1506 would prevent the cable segments 1160, 1164, 1168, 1172, 1176, 1180, 1184, 1188 from sliding relative to the cables 1012, 1014, 1022, 1024, 1032, 1034, 1042, 1044 as pulling and pushing motions are applied thereto in the manners described above.

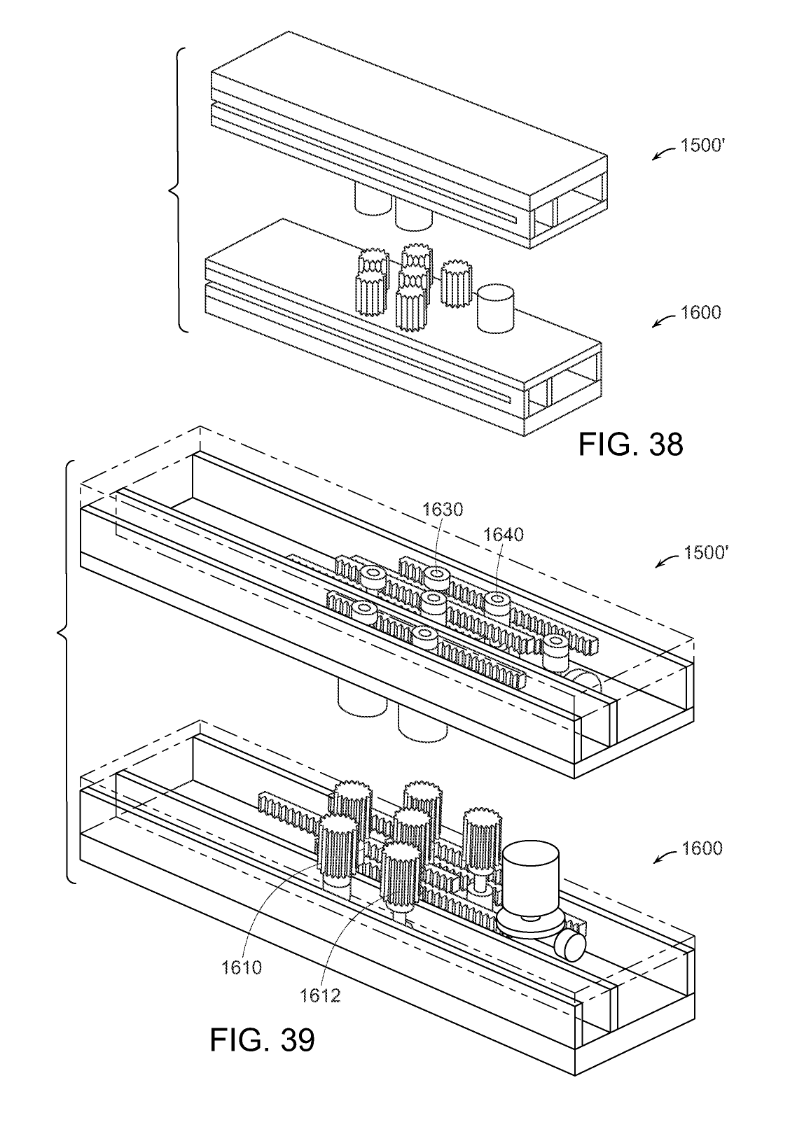

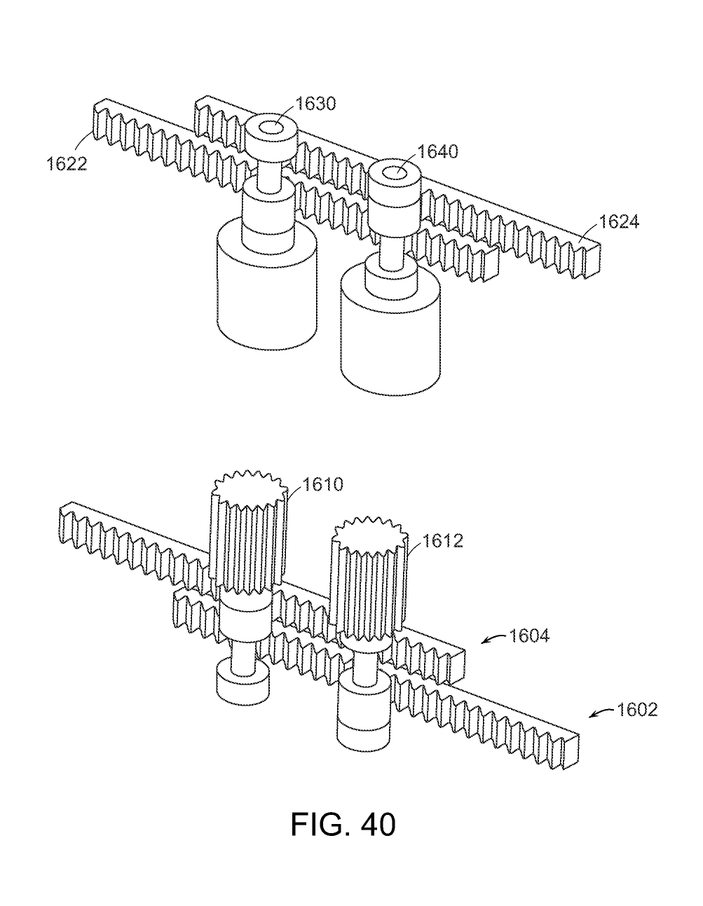

FIGS. 38-40 illustrate alternative cable coupling arrangement 1500′ that may be effectively employed in the cable docking station 1100 without the use of the various pitch racks described above. For example, in this embodiment, the docking station 1100 would include a lower gear housing 1600 that slidably supports a series of first lower gear racks 1602. Each first lower gear rack 1602 held in slidable registration with a second lower gear rack 1604. Although not shown, the distal end of each cable 1012, 1014, 1022, 1024, 1032, 1034, 1042, and 1044 is attached to a corresponding lower gear rack 1604. A pair of pinion gear assemblies 1610, 1612 correspond to each pair of first and second lower gear racks 1602, 1604. Similarly the steerable guide tube assembly 1300 would have an upper gear housing 1620 therein that slidably supports a series of first upper gear racks 1622 and second upper gear racks 1624. The first and second upper gear racks 1622, 1624 interface with a corresponding pair of pinion gear assemblies 1630, 1640 that are adapted to meshingly engage with the pinion gear assemblies 1610, 1612 when the steerable guide tube assembly 1300 is docked onto the cable docking station 1100 in the manner described above.

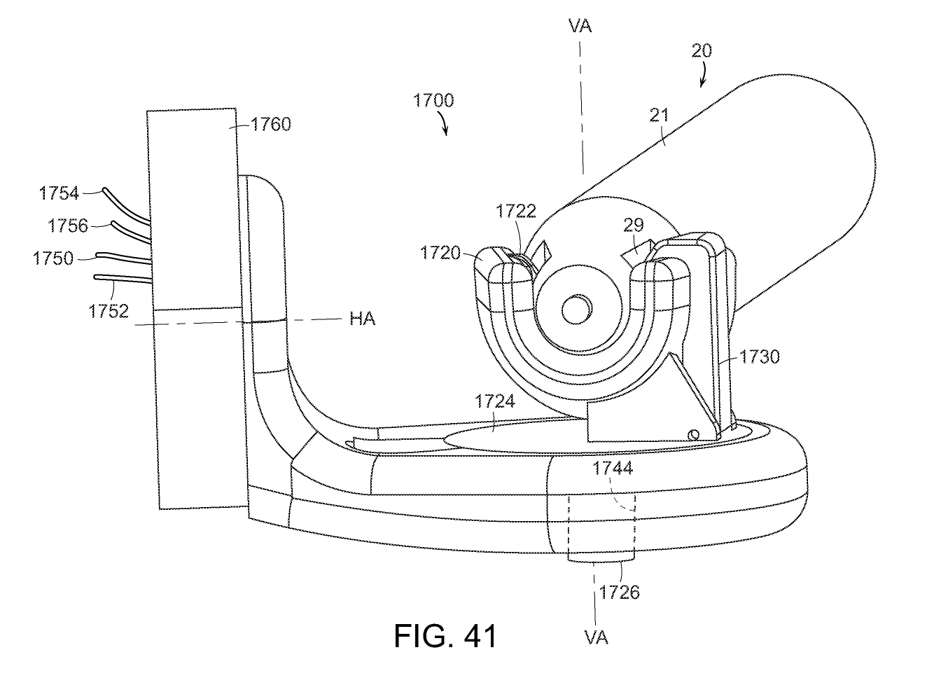

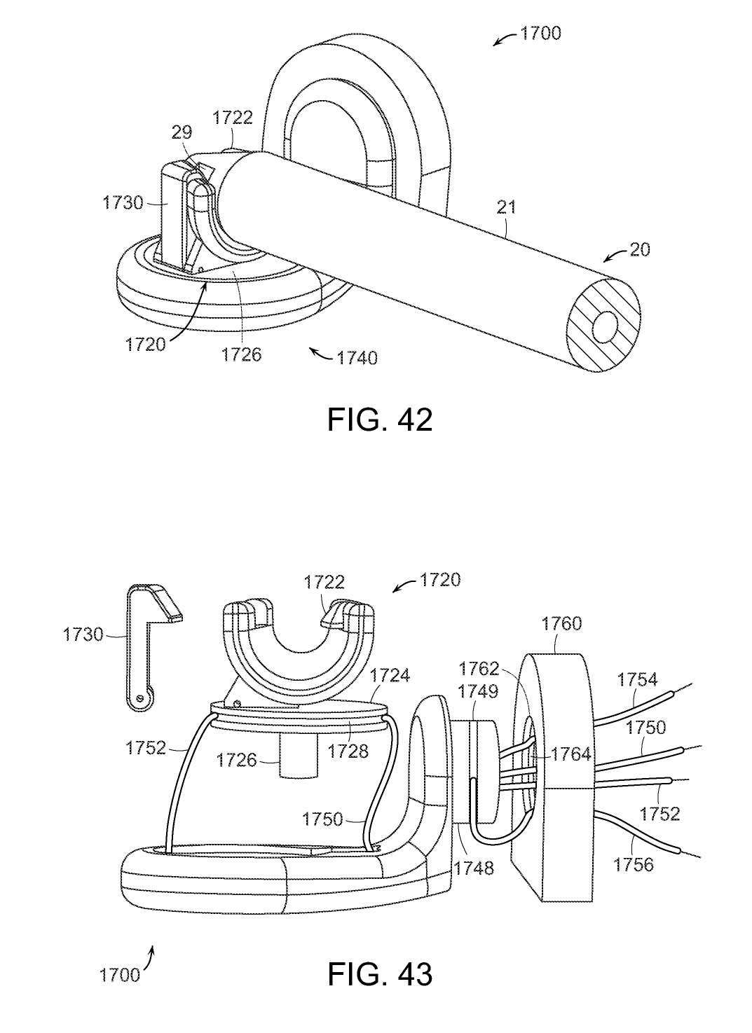

FIGS. 41-44 illustrate another flexible user interface assembly 1700 of the present invention that may be used in connection with an endoscopic surgical instrument 20. In this embodiment, the distal end portion 21 of the endoscopic surgical instrument 20 may be provided with a radial slot segment 29 that is adapted to slidably receive a corresponding retention protrusion 1722 formed on a first rotator 1720. The first rotator 1720 may further include a latch 1730 that is configured to be pivoted into the radial slot segment 29 of the surgical instrument 20 when the distal end portion 21 is mounted to the first rotator 1720 as shown in FIGS. 41 and 42. A spring (not shown) may be employed to retain the latch 1730 in the latched position, yet enable the user to pivot the latch 1730 out of the radial slot segment 29 when it is desired to remove the surgical instrument 20 from the first rotator 1720.

The first rotator 1720 may further have a circular yoke base 1724 that is sized to be received in a circular cavity 1742 in a second base 1740. A lower axle 1726 protrudes from the yoke base 1724 and is sized to be rotatably received in a hole 1744 in the second base 1740 to facilitate pivotal travel of the first rotator 1720 relative to the second base 1740 about a vertical axis VA-VA. The lower axle 1726 may protrude out of the second base 1740 and have a snap ring (not shown) or other fastener arrangement to retain the lower axle 1726 within the hole 1744 while facilitating rotation of the lower axle 1726 therein about the vertical axis VA-VA.

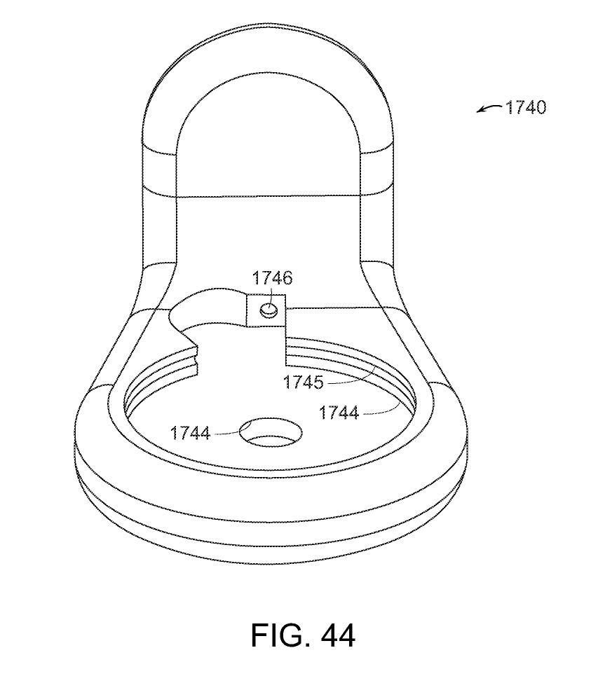

A pair of first steering cables 1750 and 1752 may be attached to the yoke base 1724 and be received in a radially formed groove 1728 in the perimeter of the yoke base 1724 and a mating groove 1745 formed around the perimeter of the cavity 1724 in the second base 1740. The steering cables 1750, 1752 may extend through a passage 1746 in the second base 1740 that further extends through an axle portion 1748 formed thereon. See FIG. 44. Axle portion 1748 is sized to be rotatably received in a hole 1762 in a base portion 1760. Base portion 1760 may comprise a stand, a portion of a bed, a mounting bracket, etc. The axle portion 1748 facilitates rotation of the second base 1740 relative to the base portion 1760 about a horizontal axis HA-HA. A pair of second steering cables 1754, 1756 may be attached to the axle portion 1748 and be received in a radially formed groove 1749 in the perimeter of the axle portion 1748 and a corresponding radial groove 1764 formed in the base portion 1760. See FIG. 43. The flexible user interface assembly 1700 may function as a “two stage gimbal arrangement for applying t control to the steerable cables 1750, 1752, 1754, 1756 attached to a steerable guide tube assembly 1300 of the type described above.

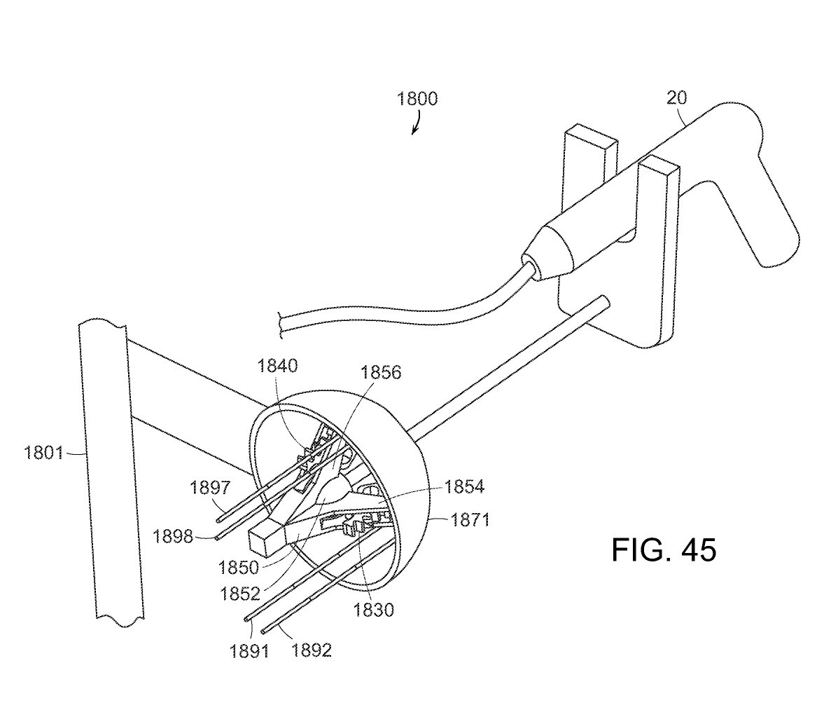

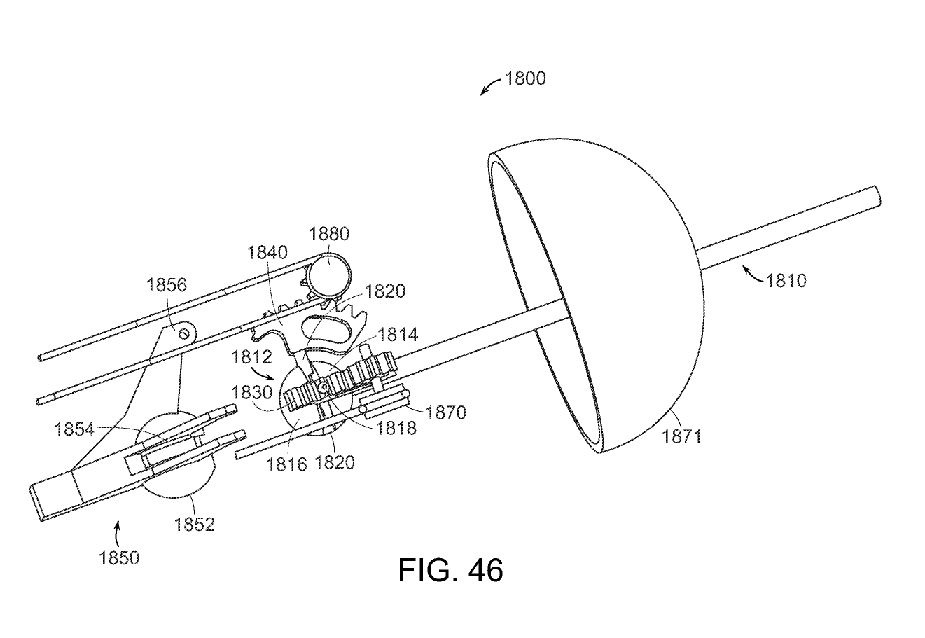

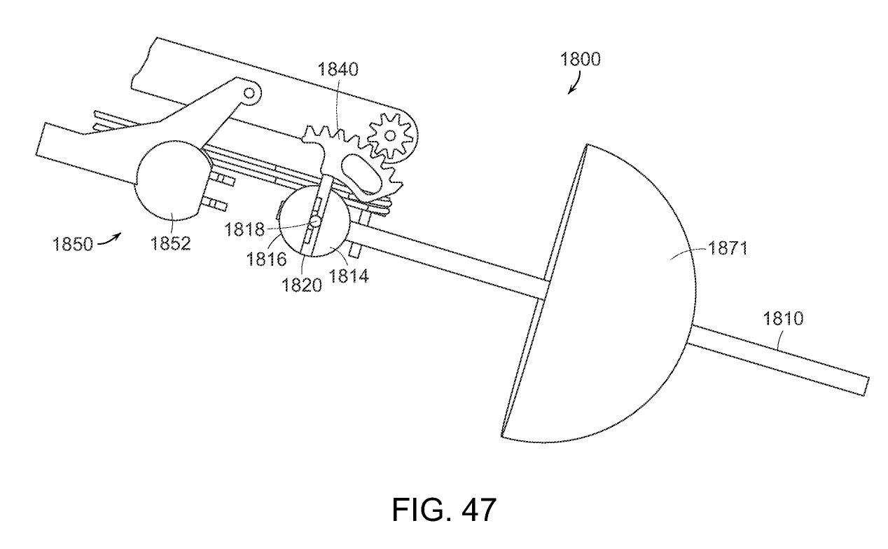

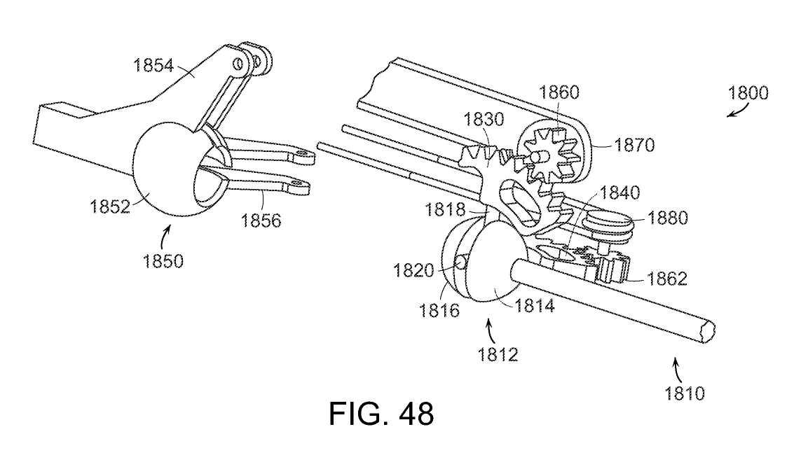

FIGS. 44-47 illustrate yet another flexible user interface assembly 1800 of the present invention. This embodiment includes a tool mounting portion or rod 1810 to which an endoscopic surgical instrument 20 may be mounted. In various embodiments, the tool mounting rod 1810 has a ball assembly 1812 formed thereon. The ball assembly 1812 may comprise a first ball segment 1814 and a second ball segment 1816. Constrained between the ball segments 1814 and 1816 is a pair of cross members 1818, 1820 that are pinned together or are otherwise nonmovably fixed to each other. A first arcuate gear segment 1830 is attached to cross member 1816 and a second arcuate gear segment 1840 is attached to the other cross member 1820 at right angles to the first arcuate gear segment 1830. See FIG. 47. The ball assembly 1812 may be movably supported in a socket assembly 1850 that is nonmovably supported or attached to a portion of the housing. FIGS. 34 and 35 illustrate a portion 1871 of the housing that may be attached to a stand, bed, etc, generally depicted as 1801 in FIG. 33.

As can be seen in FIGS. 34 and 35, the socket assembly 1850 includes a ball portion 1852 that is configured to receive the ball assembly 1812 therein. The socket assembly 1812 may further include a first set of pinion support arms 1854 for supporting a first pinion gear 1860 thereon in meshing engagement with the first arcuate gear segment 1830 and a second set of pinion arms 1856 for supporting a second pinion gear 1862 in meshing engagement with the second arcuate gear segment 1840. When assembled, the first pinion gear 1860 and the second pinion gear 1862 are positioned at right angles to each other. In various embodiments a first pulley 1870 is attached to the first pinion gear 1860 for rotation therewith a second pulley 1880 is attached to the second pinion gear 1862 for rotation therewith. Thus, rotation of the ball assembly 1812 along a first plane defined by the first arcuate gear segment 1830 will result in the rotation of the first pinion gear 1860 and rotation of the ball assembly 1812 in a second plane that is orthogonal to the first plane will result in the rotation of the second pinion gear 1862.

In various embodiments, a first cable 1890 is sheaved around the first pulley such that ends 1891 and 1892 of the cable 1890 may be operably coupled to corresponding cable segments of a steerable guide tube assembly 1300 in any of the various manners described above or otherwise used to control a steerable guide tube. For example, as the first pulley is rotated in a first direction, end 1891 may get pulled in the first direction wherein end 1892 is pushed in an opposite direction. Similarly, a second cable 1896 is sheaved around the second pulley such that ends 1897 and 1898 may be operably coupled to corresponding cable segments of a steerable guide tube assembly 1300 in any of the various manners described above or otherwise used to control a steerable guide tube assembly. As the second pulley is rotated in another first direction, end 1897 may get pulled in that another first direction and end 1898 may get pushed in the opposite direction. As such, after the ends 1891, 1892 of the first cable 1890 and the ends 1897, 1898 of the second cable have been coupled to the cable segment used to control a steerable guide tube, movement of the surgical instrument 20 along a first plane may result in the manipulation of the distal end of the guide tube, for example, in up and down directions. In addition, manipulation of the surgical instrument 20 in a second plane that is orthogonal to the first plane may result in the manipulation of the distal end of the steerable guide tube in, for example, left and right directions.

Those of ordinary skill in the art will readily appreciate that the flexible user interface support assembly embodiments of the present invention translates laparoscopic-like manipulation to linear pull-push motion. The push-pull motion enables the use of cables to generate tool-tip articulation at the end of the steerable guide tube assembly, thereby providing the clinician with a familiar laparoscopic-like user experience during the surgical procedure. Furthermore, the flexible user interface embodiments described immediately above facilitates the translation of the tool/instrument articulation motions into rotary motions. The rotary motion is then translated through the drive shafts into the pulleys. The pulleys serve to translate the rotary motion to linear translation of the cables. The cables translate along the gooseneck inside coil pipe to allow the dynamic location of the steerable guide tube assembly. In addition, the unique and novel cable docking station embodiments enables the quick coupling of a cable-controlled interface with a cable-controlled guide tube assembly, without cables hanging out of the devices to become inadvertently tangled and possibly damaged.

Those of ordinary skill in the art will appreciate that the unique and novel aspects of the various embodiments of the flexible interface support assemblies of the present invention provide the clinician with the ability to control the articulation of a working channel into which a portion of a surgical instrument has been inserted, simply by manipulating the surgical instrument relative to a fixed position. In particular, various embodiments of the present invention provide separate control of right and left working channel horizontal articulation and separate control of right and left working channel up/down articulation. While the embodiment depicted in FIGS. 1-6 above is adapted for use with two separate surgical tools or instruments, other embodiments could be constructed to support a single surgical tool, while still other could be adapted to support more than two surgical tools. The use of the friction hinges enables the clinician to pivot the tools about a corresponding fixed vertical axis and retain the tool in that position when the clinician releases the tool. The unique and novel means for connecting the cables from the steerable guide tube assembly 200 to the mounting assembly facilitate quick and easy attachment employment of the flexible interface systems with a variety of different cable driven guide tube assemblies. The mobile nature of the stand and the flexible gooseneck arrangement enables the system to be advantageously located and positioned within the surgical suite.

While the embodiments have been described, it should be apparent, however, that various modifications, alterations and adaptations to the embodiments may occur to persons skilled in the art with the attainment of some or all of the advantages of the invention. For example, according to various embodiments, a single component may be replaced by multiple components, and multiple components may be replaced by a single component, to perform a given function or functions. This application is therefore intended to cover all such modifications, alterations and adaptations without departing from the scope and spirit of the disclosed invention as defined by the appended claims.

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An interface system for aiding clinicians in controlling and manipulating at least one endoscopic surgical instrument and a cable-controlled guide tube system, said interface system comprising:
a surgical tool docking assembly supportable relative to the cable-controlled guide tube system, said surgical tool docking assembly comprising:
a cable drive assembly operably couplable to the cable-controlled guide system for applying control motions thereto; and
a first tool docking station configured to support one of the at least one endoscopic surgical instruments for selective pivotal travel about a first axis upon application of a first motion thereto and about a second axis upon application of a second motion thereto, said first tool docking station operably coupled to at least one first drive shaft for imparting a corresponding rotary drive motion to said cable drive assembly.

2. The interface system of claim 1 wherein each of the said at least one endoscopic surgical instruments supported on said first tool docking station has a flexible working portion that has a distal tip portion that communicates with the cable controlled guide tube system and wherein said interface system is configured such that movement of a handle portion of the at least one endoscopic surgical instrument supported on said first tool docking station in a first direction imparts a control motion to the cable controlled guide tube system to cause the distal tip portion thereof to move in a second direction opposite to said first direction.

3. The interface system of claim 1 wherein each of the said at least one endoscopic surgical instruments supported on said first tool docking station has a flexible working portion that has a distal tip portion that communicates with the cable controlled guide tube system and wherein said interface system is configured such that movement of a handle portion of the at least one endoscopic surgical instrument supported on said first tool docking station in a first direction imparts a control motion to the cable controlled guide tube system to cause the distal tip portion thereof to move in said first direction.

4. The interface system of claim 1 wherein said first tool docking station comprises:
a first tool mounting assembly to operably support the one of the at least one endoscopic surgical instruments;
a first ball and socket assembly operably coupled to said first tool mounting assembly;
a first drive shaft interfacing with said first ball and socket assembly and said cable drive assembly such that when said first tool mounting assembly is pivoted about said first axis, said first ball and socket assembly imparts a first rotary motion to said first drive shaft; and
another first drive shaft interfacing with said first ball and socket assembly and said cable drive assembly such that when said first tool mounting assembly is pivoted about said second axis, said first ball and socket assembly imparts another first rotary motion to said another first drive shaft.

5. The interface system of claim 4 wherein said first ball and socket assembly comprises:
a first sphere holder:
a first sphere assembly rotatably supported by said first sphere holder, said first sphere assembly operably coupled to said first tool mounting assembly;
a first driver gear segment on said first sphere assembly, said first driver gear segment in meshing engagement with a portion of said first drive shaft;
another first driver gear segment on said first sphere assembly, said another first driver gear segment in meshing engagement with a portion of said another first drive shaft; and
a first sphere holder plate movably coupled to said first sphere holder to retain said first sphere assembly therein.

6. The interface system of claim 5 wherein said first sphere holder plate is configured to establish a degree of friction between said sphere assembly and said first sphere holder plate sufficient to retain said first sphere assembly in a desired position relative to said first sphere holder upon discontinuing application of at least one of said first and second motions to said first tool docking station.

7. The interface system of claim 1 wherein said first tool docking station is further configured to support at least one of the at least one endoscopic surgical instruments for selective axial travel about a third axis upon application of a third motion thereto to impart a corresponding axial motion to a flexible working portion thereof.

8. The interface system of claim 1 wherein at least one of said at least one endoscopic surgical instruments supported on said first tool docking station has a flexible working portion comprising a flexible working member movably received within a flexible sheath communicating with said cable controlled guide tube system and wherein said interface system comprises a seal for interfacing between said at least one endoscopic surgical instrument and the flexible sheath thereof for establishing a substantially airtight seal therebetween.

9. The interface system of claim 1 wherein said first tool docking station further comprises:
a first tool docking station handle; and
a clamping member attached to said first tool docking station handle for releasably mounting the at least one endoscopic surgical instrument thereto.

10. The interface system of claim 1 wherein said surgical tool docking assembly and the cable controlled guide tube assembly are mounted to a common member.

11. The interface system of claim 1 further comprising a second docking station configured to support another one of the at least one endoscopic surgical instruments for selective pivotal travel about a third axis and a fourth axis, said second tool docking station operably coupled to at least one second drive shaft for imparting a corresponding another rotary drive motion to said cable drive assembly.

12. The interface system of claim 11 wherein said second tool docking station comprises:
a second tool mounting assembly configured to operably support the another one of the at least one endoscopic surgical instruments therein;
a second ball and socket assembly operably coupled to said second tool mounting assembly;
a second drive shaft interfacing with said second ball and socket assembly and said cable drive assembly such that when said second tool mounting assembly is pivoted about said third axis, said second ball and socket assembly imparts a second rotary motion to said second drive shaft; and
another second drive shaft interfacing with said second ball and socket assembly and said cable drive assembly such that when said second tool mounting assembly is pivoted about said fourth axis, said second ball and socket assembly imparts another second rotary motion to said another second drive shaft.

13. The interface system of claim 12 wherein said second ball and socket assembly comprises:
a second sphere holder supported relative to said cable drive assembly;
a second sphere assembly rotatably supported by said second sphere holder, said second sphere assembly operably coupled to said second tool mounting assembly;
a second driver gear segment on said second sphere assembly, said second driver gear segment in meshing engagement with a portion of said second drive shaft;
another second driver gear segment on said second sphere assembly, said another second driver gear segment in meshing engagement with a portion of said another second drive shaft; and
a second sphere holder plate movably coupled to said second sphere holder to retain said second sphere assembly therein

14. The interface system of claim 1 wherein said cable drive assembly comprises:
a first cable pulley rotatably supported relative to said first tool docking station and configured to receive said rotary drive motion from said first drive shaft;
a first upper cable coupled to said first cable pulley and operably attachable to a corresponding first portion of said cable-controlled guide tube system; and
a first lower cable coupled to said first cable pulley and operably attachable to another corresponding first portion of said cable-controlled guide tube system.

15. The interface system of claim 4 wherein said cable drive assembly comprises:
a first cable pulley rotatably supported relative to said first tool docking station and configured to receive said rotary drive motion from said first drive shaft;
a first upper cable coupled to said first cable pulley and operably attachable to a corresponding first portion of said cable-controlled guide tube system;
a first lower cable coupled to said first cable pulley and operably attachable to another corresponding first portion of said cable-controlled guide tube system;
a second cable pulley rotatably supported relative to said first tool docking station and configured to receive another said rotary drive motion from said another first drive shaft;
a second upper cable coupled to said second cable pulley and operably attachable to a corresponding second portion of said cable-controlled guide tube system; and
a second lower cable coupled to said second cable pulley and operably attachable to another corresponding second portion of said cable-controlled guide tube system.

16. The interface system of claim 11 wherein said cable drive assembly comprises:
a first cable pulley rotatably supported relative to said first tool docking station and configured to receive said rotary drive motion from said first drive shaft;
a first upper cable coupled to said first cable pulley and operably attachable to a corresponding first portion of said cable-controlled guide tube system;
a first lower cable coupled to said first cable pulley and operably attachable to another corresponding first portion of said cable-controlled guide tube system;
a second cable pulley rotatably supported relative to said first tool docking station and configured to receive another said rotary drive motion from another first drive shaft operably interfacing with said first tool docking station;
a second upper cable coupled to said second cable pulley and operably attachable to a corresponding second portion of said cable-controlled guide tube system;
a second lower cable coupled to said second cable pulley and operably attachable to another corresponding second portion of said cable-controlled guide tube system;
a third cable pulley rotatably supported relative to said second tool docking station and configured to receive a third rotary drive motion from one of said at least one second drive shaft;
a third upper cable coupled to said third cable pulley and operably attachable to a corresponding third portion of said cable-controlled guide tube system;
a third lower cable coupled to said third cable pulley and operably attachable to another corresponding third portion of said cable-controlled guide tube system;
a fourth cable pulley rotatably supported relative to said second tool docking station and configured to receive a fourth rotary drive motion from another second drive shaft operably interfacing with said second tool docking station;
a fourth upper cable coupled to said fourth cable pulley and operably attachable to a corresponding fourth portion of said cable-controlled guide tube system; and
a fourth lower cable coupled to said fourth cable pulley and operably attachable to another corresponding fourth portion of said cable-controlled guide tube system.

17. The interface system of claim 13 further comprising:
a first coupler member coupled to a distal end of said first cable;
a second coupler member coupled to a distal end of said second cable and wherein said corresponding first portion of said cable-controlled guide tube system comprises:
a first coupler member attached to a proximal end of said corresponding first guide cable and wherein said another corresponding first portion of said cable-controlled guide tube system comprises:
a first distal cable segment operably supported in said cable-controlled guide tube system and corresponding to said first cable;
another first coupler member attached to a proximal end of said first distal cable segment for mating engagement with said first coupler member;
a second distal cable segment operably supported in said cable-controlled guide tube system and corresponding to said second cable; and
another second coupler member attached to a proximal end of said second distal cable segment for mating engagement with said second coupler member.

18. An interface system for interfacing between at least one endoscopic surgical instrument and a cable-controlled guide tube system, said interface system comprising:
a first tool docking station for supporting a first endoscopic surgical instrument for selective pivotal travel about a first axis and a second axis; and
a cable drive assembly configured to operably interface with the cable-controlled guide tube system and said first tool docking station such that when said first endoscopic tool is pivoted about said first axis, said cable drive assembly imparts at least one first control motion to said cable-controlled guide tube system and when said first endoscopic tool is pivoted about said second axis, said cable drive assembly imparts at least one of other first control motions to said cable-controlled guide tube assembly.

19. The interface system of claim 18 further comprising a second docking station configured to support a second endoscopic surgical instrument for selective pivotal travel about a third axis and a fourth axis and wherein said cable drive assembly is configured to operably interface with the cable-controlled guide tube system and said second tool docking stations such that when said second endoscopic tool is pivoted about said third axis, said cable drive assembly imparts at least one second control motion to said cable-controlled guide tube system and when said second endoscopic tool is pivoted about said fourth axis, said cable drive assembly imparts at least one of other second control motions to said cable-controlled guide tube assembly.

20. A cable docking station for interfacing between a cable drive system and a cable-controlled guide tube system, said cable docking station comprising:
a support member configured to dockingly interface with a portion of the cable-controlled guide tube system; and
a proximal cable coupler attached to a distal end of a first cable extending from the cable drive system, said proximal cable coupler operably supported within said support member such that when said support member is docked with said portion of the cable-controlled guide tube system, said proximal cable coupler drivingly engages a corresponding distal cable coupler attached to a corresponding first distal cable segment in the cable-controlled guide tube system.

21. The cable docking station of claim 20 wherein said proximal cable coupler comprises a first pitch rack and wherein said distal cable coupler comprises a first distal pitch rack configured to meshingly engage the first pitch rack when said support member is docked with said portion of the cable-controlled guide tube system.

22. The cable docking system of claim 20 wherein said proximal cable coupler comprises a first retention member attached to a distal end of said first cable and wherein said distal cable coupler comprises a first distal retention bead on a proximal end of said first distal cable segment such that said retention bead is retainingly engaged within a first slot in said first distal retention member when said support member is docked with said portion of the cable-controlled guide tube system.

23. The cable docking system of claim 20 further comprising:
a first gear housing supported on said support member;
a first gear rack slidably supported on said first gear housing and coupled to a distal end of said first cable;
a first pinion gear assembly in meshing engagement with said first gear rack and wherein said cable docking system further comprises:
a second gear housing on the cable-controlled guide tube system;
a second gear rack slidably supported on said second gear housing and attached to said first distal cable segment; and
a second pinion gear assembly in meshing engagement with said second gear rack and configured for meshing engagement with said first pinion gear assembly when said support member is in docking engagement with said portion of said cable-controlled guide tube system.

24. An interface system for aiding clinicians in controlling and manipulating at least one endoscopic surgical instrument and a cable-controlled guide tube system, said interface system comprising:
a base;
a second base rotatably attached to said base for selective rotation relative thereto about a first axis;
a first rotator rotatably supported on said second base for selective rotation relative thereto about a second axis, said first rotator configured to releasably support the endoscopic surgical instrument therein;
at least one first steering cable attached to said first rotator and coupled to a portion of the cable-controlled guide tube system such that rotation of said first rotator causes said at least one first steering cable to provide at least one actuation motion to the cable-controlled guide tube system.

25. The interface system of claim 24 further comprising at least one pitch steering cable attached to said second base and coupled to a portion of the cable-controlled guide tube system such that rotation of said second base causes said at least one pitch steering cable to provide at least one other actuation motion to the cable-controlled guide tube system.

26. The interface system of claim 24 further comprising a latch system for releasably latching the endoscopic surgical instrument to said first rotator.

27. The interface system of claim 26 wherein said latch system comprises:
a latch member movably coupled to said first rotator and being pivotable between a latched position wherein the latch retainingly engages a portion of the endoscopic surgical instrument supported in said first rotator and an unlatched position; and
a biasing member for biasing the latch member into the latched position.
